(19) United States
(12) Patent Application Publication
(10) Pub. No.: US 2009/0181901 A1
(43) Pub. Date: Jul. 16, 2009

(54) COMPOSITIONS AND METHODS TO INCREASE BIOAVAILABILITY OF CAROTENOIDS

(76) Inventor: Thomas Eidenberger, Steyr (AT)

Correspondence Address: FULBRIGHT & JAWORSKI L.L.P.;Attn: MN IP Docket, 600 Congress Avenue, Suite 2400, Austin, TX, 78701, US

(21) Appl. No.: 12/270,231

(22) Filed: Nov. 13, 2008

Related U.S. Application Data
(60) Provisional application No. 60/987,866, filed on Nov. 14, 2007.
(60) Provisional application No. 61/037,191, filed on Mar. 17, 2008.

(51) Int. Cl. A61K 31/045; A61K 31/197; A61K 38/06; A61K 31/047; A61K 31/122; A61K 31/336

(52) U.S. Cl. 514/18; 514/562; 514/729; 514/691; 514/475

(57) ABSTRACT

The present invention relates to compositions and methods of use to increase the bioavailability of a carotenoid in a subject. The compositions include a carotenoid and either an anthocyanin or a compound having at least one —SH group or combinations of both an anthocyanin and a compound having at least one —SH group and mixtures thereof. The compositions provide that when the composition is administered, the resulting bioavailability of the carotenoid is increased relative to a carotenoid composition that is without either the anthocyanin, is without the compound having at least one —SH group or is without the combination of an anthocyanin/a compound having at least one —SH group and mixtures thereof.

### CROSS-REFERENCE TO RELATED APPLICATIONS

This application claims benefit of U.S. Provisional Application No. 60/987,866 filed on Nov. 14, 2007 and U.S. Provisional Application No. 61/037,191 filed on Mar. 17, 2008, the contents of each of which are herein incorporated by reference.

### FIELD OF THE INVENTION

The invention relates generally to compositions and methods to provide increased amounts of bioavailable carotenoids, such as xanthophylls, including lutein, zeaxanthin and related compounds and/or an anthocyanin extract composition that includes an anthocyanin extract and/or a stabilizing compound having at least one —SH group.

### BACKGROUND OF THE INVENTION

Carotenoids are yellow, red and orange pigments that are widely distributed in nature. Although specific carotenoids have been identified in various fruits and vegetables, bird feathers, egg-yolk, poultry skin, crustaceans and macular eye region, they are especially abundant in marigold petals, corn and leafy vegetables. The correlation between dietary carotenoids and carotenoids found in human serum and plasma indicate that only selected groups of carotenoids make their way into the human blood stream to exert their effect.

Carotenoids absorb light in the 400-500 nm region of the visible spectrum. This physical characteristic imparts the yellow/red color to the pigments. Carotenoids contain a conjugated backbone composed of isoprene units, which are usually inverted at the center of the molecule, imparting symmetry. Changes in geometrical configuration about the double bonds result in the existence of many cis- and trans-isomers. Mammalian species do not synthesize carotenoids and therefore these have to be obtained from dietary sources such as fruits, vegetables and egg yolks. In the recent years, carotenoids have been attributed several health benefits, which include prevention and or protection against serious health disorders.

Carotenoids are non-polar compounds classified into two sub-classes, namely more polar compounds called xanthophylls or oxy-carotenoids and non-polar hydrocarbon carotenes like [beta]-carotene, lycopene, etc. Both the sub-classes have at least nine conjugated double bonds responsible for the characteristic color of the carotenoids. Xanthophylls have ring structures at the end of the conjugated double bond chain with polar functionalities, such as hydroxyl or keto groups. Examples of xanthophylls include lutein, zeaxanthin, capsanthin, canthaxanthin, β-cryptoxanthin, astaxanthin, etc. As natural colorants and also for their role in human health, xanthophylls containing lutein and zeaxanthin have attracted the renewed attention of scientists and researchers in the biomedical, chemical and nutritional field in recent years.

Lutein and zeaxanthin contribute to yellow and orange-yellow color respectively. Lutein and zeaxanthin can be present in plant material in free form (non-esterified) and also as esters. Lutein is present in green leafy vegetables like spinach, kale and broccoli in the free form while fruits like mango, orange, papaya, red paprika, algae and yellow corn. These sources generally contain lutein in the form of its fatty esters. Lutein is also present in the blood stream and various tissues in human body and particularly the macula, lens and retina of the eye.

Marigold (Tagetes erecta) flower petals are a rich source of lutein in its esterified form. The ester portion(s) are fatty acids. Dried marigold flowers contain approximately 1-1.6% carotenoids by weight and lutein esters content accounts for 90% of the total carotenoids. The xanthophyll fatty acid esters composition in marigold oleoresin chiefly consists of lutein in its ester form as di-palmitate, myristate-palmitate, palmitate-stearate, dimyristate and monoesters.

Lutein obtained by the hydrolysis of lutein esters from marigold has been found to be identical to the lutein found in fruits, vegetables and in human plasma and the macular region. After absorption, the human body cannot distinguish the source of lutein. Therefore, a widely cultivated and commercially processed raw material like marigold, which is already used by the food and feed industry, is an attractive source for lutein in view of abundant availability and cost considerations.

Essentially, lutein esters and lutein in the free form are commercially important nutraceuticals obtained from marigold flowers. Dried flowers are used for obtaining marigold extract or oleoresin. By subjecting the extract/oleoresin to saponification, xanthophylls in the free form are obtained. The resultant alkali salts of fatty acids obtained from the saponification are removed and the xanthophyll containing mixture of lutein and zeaxanthin purified further.

In the fresh marigold flowers, lutein esters exist in trans-isomeric form, whereas exposure to heat, light, oxygen, acid, etc. catalyses isomerization from trans- to cis-lutein geometric isomeric forms. As a nutraceutical and food additive, the trans-isomeric form of lutein is preferred because of better bioavailability and deeper yellow color compared to the corresponding cis-isomeric form.

The free form of lutein (de-esterified) is unstable against the effects of heat and light, and also unstable at low pH values. These can lower the uptake of lutein when administered.

Therefore, a need exists to provide increase amounts of bioavailable carotenoids to individuals in need thereof.

### BRIEF SUMMARY OF THE INVENTION

The present invention surprisingly provides the ability to provide increased amounts of bioavailable carotenoids, such as a xanthophyll (e.g., lutein) or a carotene by including either a fruit extract containing an anthocyanin extract and/or a stabilizing compound having at least one —SH group (e.g., cysteine or glutathione) in a stabilized composition (relative to compositions that do not include either an anthocyanin extract and/or a stabilizing compound having at least one —SH group). The compositions and methods provide that the carotenoid's bioavailability is increased within a subject in need thereof, by at least about 15 percent to about 4 times the amount of bioavailable carotenoid relative to samples provided that do not include either an anthocyanin, a compound having at least one —SH group or combinations thereof.

Suitable examples of hydroxyl containing xanthophylls include, but are not limited to, lutein, zeaxanthin, capsanthin, β-cryptoxanthin, astaxanthin, antheraxanthin, diatoxanthin, 7,8-didehydroastaxanthin, fucoxanthin, fucoxanthinol, lactucaxanthin, neoxanthin, peridinin, siphonaxanthin, violaxanthin, etc.

Additionally, the some xanthophylls contain enolizable ketone, such as for example, canthaxanthin, alpha-carotene, etc., that can be reacted in the enol form with a carboxylic acid derivative such that the enol is captured by the carboxylic acid derivative to form an enolate. A suitable example of such xanthophyll containing ketones include canthaxanthin.

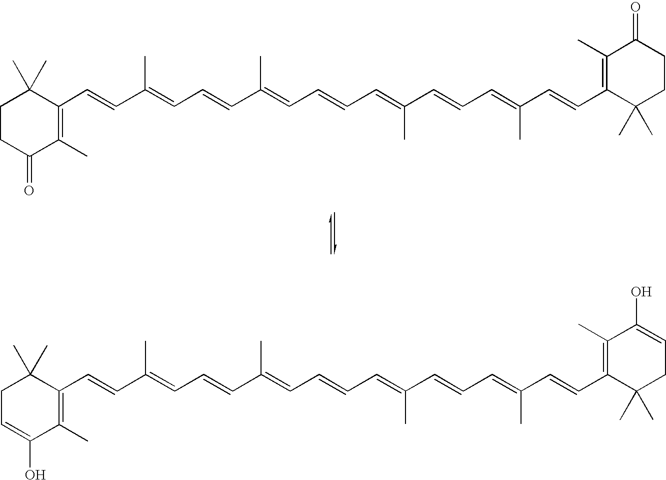

In still yet another embodiment, the present invention provides a method of preventing or inhibiting free radical oxidation, or increasing the bioavailability of a carotenoid in a mammal, the method comprising administering an effective amount of a composition as described herein.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 demonstrates the effect of cysteine dosages.

FIG. 2 demonstrates the effect of bilberry extract dosages.

FIG. 3 provides comparative cellular uptake of lutein in the presence/absence of bilberry.

FIG. 4 provides comparative cellular uptake of lutein ester in the presence/absence of bilberry.

FIG. 5 provides cellular uptake of lutein with/without black currant (upper line is with black currant and lower line is without black currant).

FIG. 6 provides cellular uptake of zeaxanthine with/without black currant (upper line is with black currant and lower line is without black currant).

### DETAILED DESCRIPTION

The present invention relates to compositions and methods of use to increase the bioavailability of a carotenoid in a subject. The compositions include a carotenoid and either an anthocyanin or a compound having at least one —SH group or combinations of both an anthocyanin and a compound having at least one —SH group and mixtures thereof. The compositions provide that when the composition is administered, the resulting bioavailability of the carotenoid is increased relative to a carotenoid composition that is without either the anthocyanin, is without the compound having at least one —SH group or is without the combination of an anthocyanin/a compound having at least one —SH group and mixtures thereof.

In general, the ratio of an anthocyanin/to a carotenoid in the compositions of the invention range from about 1 to about 500:to about 1 (by weight “w/w”).

In general, the ratio of an —SH containing compound/to a carotenoid in the compositions of the invention range from about 0.1 to about 50:about 1 (w/w).

In general, the ratio of an anthocyanin/to an —SH containing compound/to a carotenoid range from about 1 to about 500:from about 0.1 to about 50:to about 1 (w/w/w).

In general, the bioavailability of the carotenoid is increased by at least about 2 times the amount of bioavailable carotenoid relative to a control sample without either the anthocyanin or the compound having at least one —SH group or the combination of an anthocyanin and a compound having at least one —SH group. More particularly, the bioavailability of the carotenoid is increase at least about 4 times, more particularly at least about 10 times, and most particularly at least about 20 times relative to a control sample without either the anthocyanin or the compound having at least one —SH group or the combination of an anthocyanin and a compound having at least one —SH group.

In the specification and in the claims, the terms “including” and “comprising” are open-ended terms and should be interpreted to mean “including, but not limited to . . . . ” These terms encompass the more restrictive terms “consisting essentially of” and “consisting of:”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Carotenoids are a class of hydrocarbons (carotenes) and the corresponding oxygenated derivatives are xanthophylls. They consist of eight isoprenoid units joined in such a manner that the arrangement of isoprenoid units is reversed at the center of the molecule so that the two central methyl groups are in a 1,6-position relationship and the remaining nonterminal methyl groups are in a 1,5-position relationship. All carotenoids may be formally derived from the acyclic C₄₀H₅₆ structure (I) (Compound I), having a long central chain of conjugated double bonds, by (1) hydrogenation, (2) dehydrogenation, (3) cyclization, or (4) oxidation, or any combination of these processes. The class also includes compounds that arise from certain rearrangements or degradations of the carbon skeleton (I) (lycopene), provided that the two central methyl groups are retained.

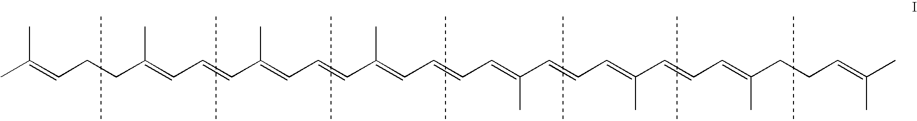

About 600 carotenoids have been isolated from natural sources. These carotenoids have been listed with their trivial and semisystematic names in Key to Carotenoids (Pfander, 1987) and in the Appendix of Carotenoids, Volume 1A (Kull & Pfander 1995) which also includes literature references for their spectroscopic and other properties. The structure is still uncertain for many of the carotenoids, including stereochemical assignments. In the cases where the structure is uncertain, resolution, followed by structural elucidation with modern spectroscopic methods (including high resolution nuclear magnetic resonance (NMR) spectroscopy) is necessary. About 370 of the naturally occurring carotenoids are chiral, bearing from one to five asymmetric carbon atoms, and in most cases one carotenoid occurs only in one configuration in Nature.

All specific names of carotenoids are based on the stem name carotene, which corresponds to the structure and numbering as in Compound II (carotene).

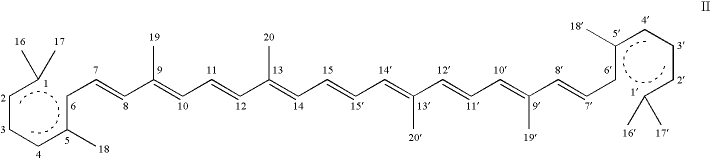

The name of a specific compound is constructed by adding two Greek letters as prefixes (Compound fragments 3) to the stem name carotene. The Greek letter prefixes are cited in alphabetical order noted in compounds IIa.

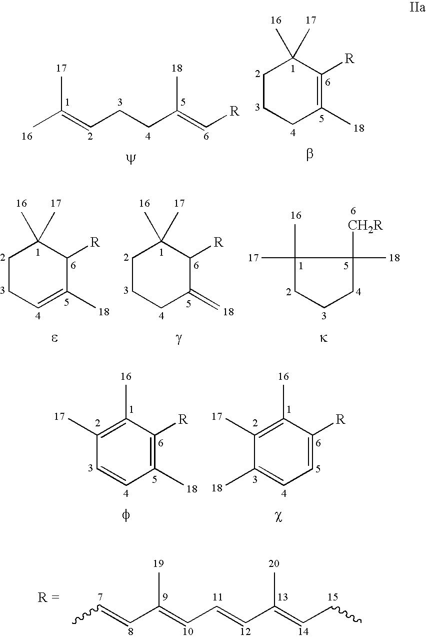

The oxygenated carotenoids (xanthophylls) most frequently include hydroxy, methoxy, carboxy, oxo, and epoxy functionality. Important and characteristic carotenoids (Compounds III through X) are lycopene (gamma, gamma-carotene) (I), beta-carotene (beta, beta-carotene) (III), alpha-carotene ((6′R)-beta, epsilon-carotene) (IV), beta-cryptoxanthin ((3R)-beta,beta-caroten-3-ol) (V), zeaxanthin ((3R,3′R)-beta, beta carotene-3,3′-diol) (VI), lutein (“xanthophyll”, (3R,3′R,6′R)-beta, epsilon-carotene-3,3′-diol) (VII), neoxanthin ((3S,5R,6R,3′S,5′R,6′S)-5′,6′-epoxy-6,7-didehydro-5,6,S′,6′-tetrahydro-beta,beta-carotene-3,5,3′-triol) (VIII), violaxanthin ((3S,5R,6R,3′S,5′R,6′S)-5,6,5′,6′-diepoxy-5,6,5′,6′-tetrahydro-beta,beta-carotene-3,3′-diol) (IX), ficoxanthin ((3S,5R,6S,3′S,5′R,6′R)-5,6-epoxy-3,3′,5′-trihydroxy-6′,7′-didehydro-5,6,7,8,5′,6′-hexahydro-beta,beta-caroten-8-one 3′-acetate) (X), canthaxanthin (beta,beta-carotene-4,4′-dione) (XI), astaxanthin ((3S,3′S)-3,3′-dihydroxy-beta,beta-carotene-4,4′-dione) (XII), beta-apo-8′-carotenal (8′-apo-beta-caroten-8′-al) (XIII) and peridinin ((3S,5R,6R,3′S,5′R,6′R)-epoxy-3,5,3′-trihydroxy-6,7-didehydro-5,6,5′,6′-tetrahydro-10,11,20-trinor-beta,beta-caroten-19′,11′-olide 3-acetate) (XIV).

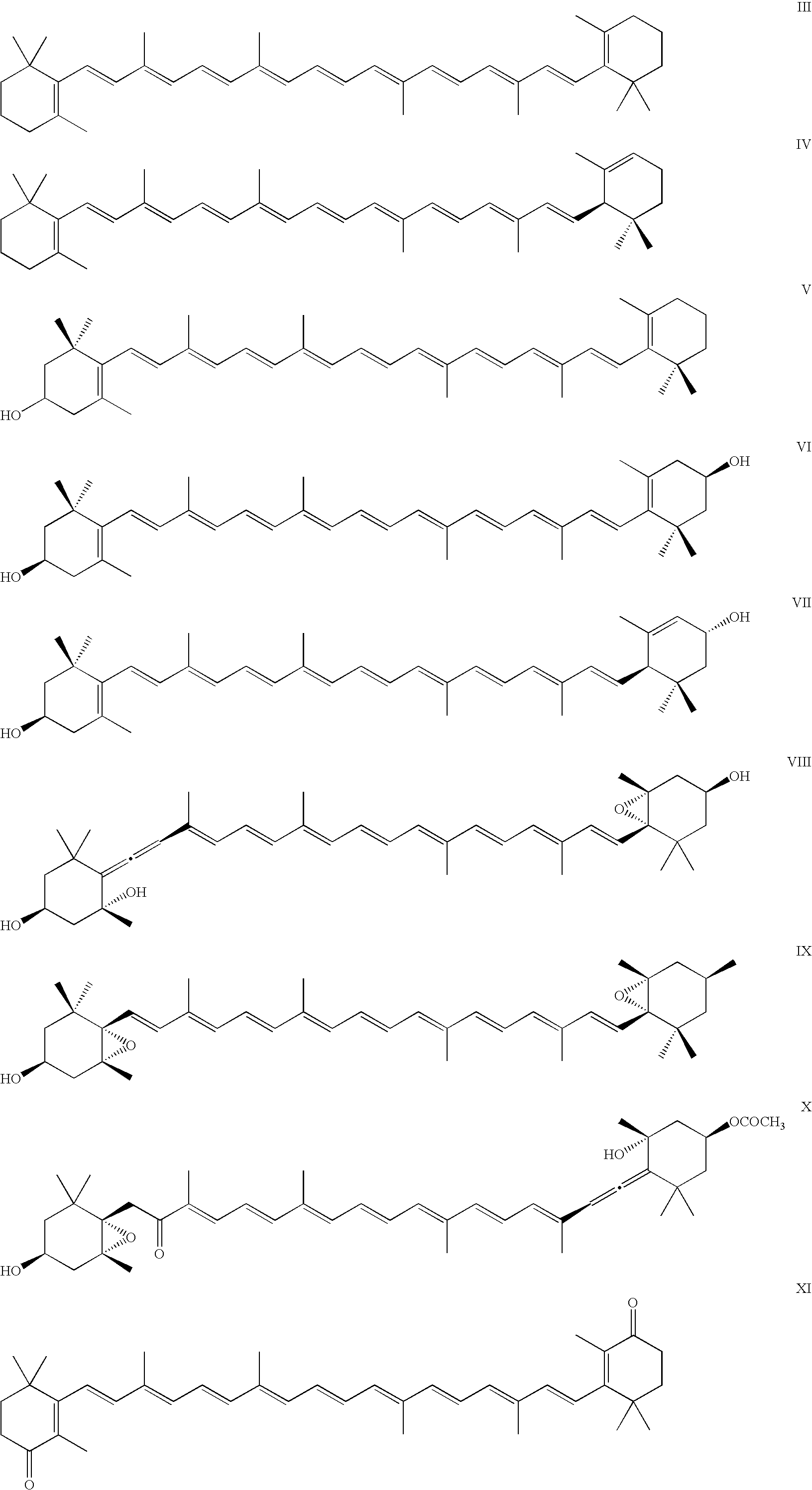

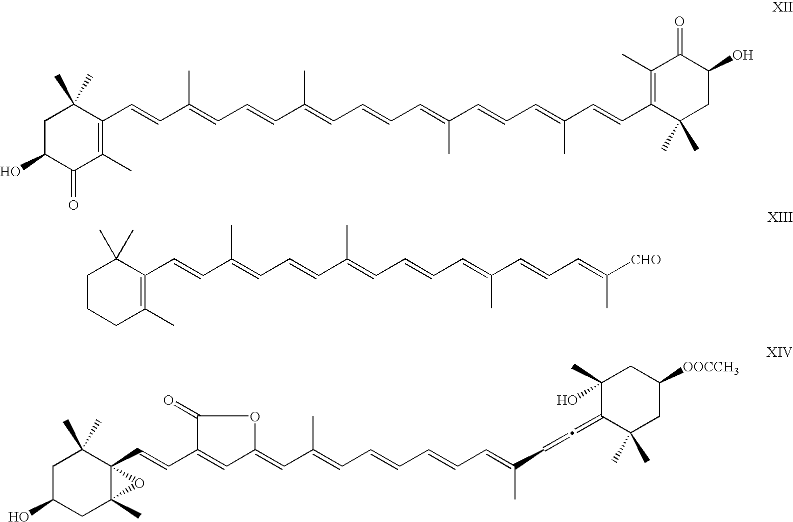

Normally carotenoids occur in Nature as the (all-E)-isomer. Some carotenoids undergo isomerization very easily during processing. For processing, it must be kept in mind that (E/Z)-isomerization can occur when a carotenoid is kept in solution. Normally the percentage of the (Z)-isomers is rather low, but it is enhanced at higher temperatures. Furthermore, the formation of (Z)-isomers is increased by exposure to light.

In commercial practice, xanthophylls of food grade quality and free of Z-lutein isomers are seldom achieved because of lack of selectivity in the raw material and improper processing conditions including high temperature drying. This results in the formation of xanthophylls of food grade quality but having higher levels of Z-lutein.

Humans and animals cannot synthesize xanthophylls like lutein and zeaxanthin, and the source of this has to be from diet. The occurrence of lutein and zeaxanthin in the macula has specific functions, viz., protection of the cells and tissues from ultra-violet light and reduced cataract risk. Lutein and zeaxanthin are known to comprise the macular pigment and lutein isomerizes into zeaxanthin in the macula.

There is evidence suggesting that lutein may have a protective effect against cancers of the breast, colon, lung, skin, cervix and ovaries and could bear promise in treatment of cardiovascular disease. Therefore, providing lutein to an individual for use in their diet or as nutritional supplements supports better human health and healthy vision.

Therefore, there is a high demand for xanthophyll materials for use as antioxidants, prevention of cataract and macular degeneration, as lung cancer-preventive agents, as agents for the absorption of harmful ultra-violet light from the rays of the sun and quencher of photo-induced free radical and reactive oxygen species, etc.

The term “xanthophyll ester” is intended to include the mono-. di-, or tri- (or more) esters of “free” xanthophyll. Typically the plant source contains the xanthophyll in the esterified form as a mono- or di-C12-C18 long chain, fatty acid such as lauric, myristic, oleic, linolenic and/or palmitic acids. Lutein in marigold flowers, zeaxanthin in wolfberries and capsanthin and capsorubin in pepper plants are present as xanthophyll diesters. The free or non-esterified xanthophyll can be found in other plants such as spinach, broccoli, kale and corn.

The term “free xanthophyll” (or free lutein, etc.) is intended to mean a carotenoid having a hydroxyl portion that remains after hydrolysis of the xanthophyll ester.

The phrase “partially hydrolyzed xanthophyll” is intended to mean a xanthophyll material originally exists as in fatty acid ester form (having as many as 3 or more fatty acid residues) that has been treated such that one or more (preferably all) of the fatty acid esters of the xanthophyll have (has) been hydrolyzed from the xanthophyll to provide a xanthophyll material that has at least one free hydroxyl group, and in particular, all fatty acid residues hydrolyzed from the xanthophyll, thereby providing a xanthophyll that is partially or fully hydrolyzed. Therefore, there is at least one hydroxyl group that is available for esterification with a carboxylic acid derivative.

It should be understood that throughout the specification an claims, when reference is made to a carotenoid, such as a xanthophyll, the term includes esterified, partially esterified and free forms of the carotenoid and should not be limiting unless otherwise noted.

The present invention relates to compositions containing one or more anthocyanins, or one or more stabilizing compounds having at least one —SH group or mixtures thereof and a carotenoid. The compositions are thus “stabile” in that the carotenoid does not readily degrade over a given period of time.

The term “anthocyanin” as used herein is intended to include both glycosylated anthocyanins (anthocyanosides) as well as the aglycon of the anthocyanoside (anthocyanidin). Throughout the specification, reference to the aglyconic anthocyanidin will often be made but should in no way be construed as limiting unless otherwise noted. Wherein either term is used, unless otherwise noted, the terms are used interchangeably and are intended to include the glycosylated as well as aglyconic materials.

Anthocyanidines, the aglyconic component of anthocyanins, have a basic structure as shown in Formula II

Formula II

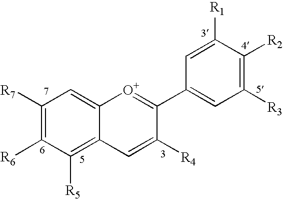

| Antho- | | | | | | | |
|---|---|---|---|---|---|---|---|
| cyanidin | R₁ | R₂ | R₃ | R₄ | R₅ | R₆ | R₇ |
| Auantinidin | H | OH | H | OH | OH | OH | OH |
| Cyanidin | OH | OH | H | OH | OH | H | OH |
| Delphinidin | OH | OH | OH | OH | OH | H | OH |
| Europinidin | OCH₃ | OH | OH | OH | OCH₃ | H | OH |
| Luteolinidin | OH | OH | H | H | OH | H | OH |
| Pelargonidin | H | OH | H | OH | OH | H | OH |
| Malvidin | OCH₃ | OH | OCH₃ | OH | OH | H | OH |
| Peonidin | OCH₃ | OH | H | OH | OH | H | OH |
| Petunidin | OH | OH | OCH₃ | OH | OH | H | OH |
| Rosinidin | OCH₃ | OH | H | OH | OH | H | OCH₃ |

where R₁ through R₇ provide representative examples of anthocyanidins.

The glycosylated forms of anthocyanins are more water soluble and stable than anthocyanidins. Anthocyanosides are classified by the number of glycosyl units they contain. Monoglycosides include one saccharidic moiety, which is primarily attached to the 3-hydroxyl group of the aglycon. Diglycosides generally contain two monosaccharides at the 3 and 5 hydroxy positions and occasionally at the 3 and 7 hydroxyl positions. Triglycosides have attachment generally where there are two units at the 3 position and one at the C-5 or C-7 position. Glycosylations at the 3′, 4′ and/or 5′ positions are also possible.

The most common sugars of anthocyanins include the monosaccharides glucose, rhamnose, galactose, arabinose and xylose. The di- and trisaccharides found most often in anthocyanins are rutinose, sophorose, sambubiose and glucorutinose.

Anthocyanins can be acylated through one or more hydroxyls with a carboxylic acid. The acids are most commonly linked to the 6 position of the monosaccharide but the 2, 3 and 4 positions of the monosaccharides are also possible. Common aliphatic acids include malonic, acetic, malic, succinic, and oxalic acids. Common aromatic phenolic acids and aliphatic dicarboxylic acids include coumaric, acffeic, sinapic, ferulic, oxalic, malonic, malic, succinic and gallic acid.

The term “extract” is intended to mean anthocyanin materials obtained from plant sources, such as leaves, twigs, bark, roots, stem, seeds, flowers, berries, fruit, for example, by routine isolation methods from suitable plants sources noted, but not limited to, those described herein. There are various methods for the extraction of anthocyanins known to those of skill in the art. Some of these methods are described in, for example, U.S. Pat. No. 5,817,354;U.S. Pat. No. 5,200,186; U.S. Pat. No. 5,912,363; U.S. Pat. No. 4,211,577; U.S. Pat. No. 4,302,200 (each incorporated herein by reference).

Examples of suitable anthocyanin-containing plants include, but are not limited to, fruits, vegetables, flowers and other plants selected from the group consisting of Acer macrophyllum, Acer platanoides, acerola, Ajuga reptans, apple, apricot, Artict bramble, avocado, banana, barberry, barley, Begonia semperfiorens, Bellis perennis, Bletilla striata, bilberry, black beans, black soybeans, black, blue and purple potatoes, blackberry, blueberry, bog whortleberry, boysenberry, buckwheat, cacao, Camellia sinensis, canarygrass, Caucasian blueberry, Chimonanthus praecox, celery, Cerasus avium, cherry, cherry laurel, chicory, chive, chokeberry, Cornelian cherry, cornflower, cotoneaster, cowberry, cranberry, crowberry, chrysanthemum, Cynomorium coccineum, Dahlia variabilis, danewort, deerberry, Dendrobium, dwarf dogwood, Echinacea purpea, eggplant, elderberry, fababean, Fatsia japonica, feijoa, fig, garlic, gerbera, ginseng, Globe artichoke, gooseberry, grapes, guava, hawthorn, hibiscus or roselle, Hibiscus Sabdaiffa, highbush blueberry, hollyhock, honeysuckle, Ipomoea purpurea, Iris ensata, Java plum, Jerusalem artichoke, kokum, Laeliocattleya, lentil, loganberry, lupine, lychee, maize, mango, mangosteen, maqui, Matthiola incana, meconopsis, Metrosideros excelsa, millet, mountain ash berry, mulberry, myrtle berry, olive, onion, orange, ornamental cherry, passion fruit, pea, peach, peanut, pear, perilla, petunia, Phalaenopsis, Phalsa, Pharbitis, Pineapple, pistachio, plum, pomegranate, Phragmites australis, purple carrot, quince, rabbiteye blueberry, radish, red and black currant, red and black raspberry, red cabbage, rice, rhubarb, rosehip, rye, saffron, sarracenia, sheepberry, Sophronitis coccinea, sorghum, sparkleberry, strawberry, Fragada Vesca, sugarcane, sunflower, sweet cherry, sweet potato, tamarillo, tamarind, taro, tart cherry, Tulip greigii, turnip, water lily, Weigela, wheat, wild rice, Verbena hybrida, yam and mixtures thereof.

Although there are literally thousands of anthocyanin extracts, all of which should be considered included within the realm of this specification, suitable examples of anthocyanin extracts of particular interest include bilberry extract, blackcurrant extract, cranberry extract, black soybean extract, cowberry extract, blueberry extract and mixtures of two or more thereof.

Typically the extract is concentrated by various methods to provide a solution enriched in anthocyanins. For example, ultrafiltration can be used to remove unwanted components by molecular weight cut offs. The retentate from the filtration can be stored as a liquid or, for example, can then be further concentrated into a powder by spray drying, freeze drying, flash drying, fluidized bed drying, ring drying, tray drying, vacuum drying, radio frequency drying or microwave drying. Ultimately, the extract should contain at least 10% by weight anthocyanin content. Commercially available anthocyanins can be obtained from sources such as Artemis International, Fort Wayne, Ind. Commercially obtained anthocyanin extracts should contain at least 10% by weight anthocyanin content. The extracts, therefore, contain anthocyanin(s) and other plant materials such as other flavinoids, sugars, etc.

Anthocyanin extracts can be further purified by one or more methods known in the art, such as chromatography, gel chromatography, high performance liquid chromatography, crystallization, affinity chromatography, partition chromatography and the like. Identification of the particular anthocyanin(s) can be accomplished by methods know to those skilled in the art and include 1H NMR, chemical degradation, chromatography and spectroscopy, especially homo- and heteronuclear two-dimensional NMR techniques for the characterization of the isolated anthocyanin compounds.

The term “purified” or “isolated” is used in reference to the purification and/or isolation of one or more anthocyanins from an anthocyanin extract as described above. Again using conventional methods known in the art, various components of the anthocyanin extract can be separated into purified materials. In one aspect of the invention, the anthocyanin(s) of the extract are substantially purified and isolated by techniques known in the art. The purity of the purified compounds is generally at least about 90%, preferably at least about 95%, and most preferably at least about 99% and even more preferably at least about 99.9% (e.g. about 100%) by weight.

In accordance with the present invention, the anthocyanin extract contains one or more anthocyanins and/or anthocyanidins selected from the group consisting of peonidin, cyanidin, pelargonidin, delphinldin, petunidin, malvidin, apigenindin, auratinidin, capensinidin, europinidin, hirsutidin, 6-hydroxycyanidin, luteolinidin, 5-methylcyanidin, pulchellidin, rosinidin, tricetnidin, derivatives and mixtures thereof. In one embodiment, the anthocyanins and anthocyanidins are selected from the group consisting of cyanidin, peonidin, malvidin, petunidin, delphinidin, their glycoside derivatives, and mixtures thereof. In yet another embodiment, the extract contains at least one cyanidin-based anthocyanin.

Anthocyanins that can be useful in the inventions described herein include, but are not limited to, cyanidin-3-glucoside; cyanidin 3-glucosylrutinoside; cyanidin-3-gentibioside; cyanidin-3-rutinoside, cyanidin-3-sambunigrin, cyanidin-3-samb-5-glucoside, cyanidin-3-galactoside, peonidin-3-rutinoside, peonidin-3-glucoside, peonidin-3-galactoside, peonidin, cyanidin, cyanidin-3 sophoroside, pelargonidin, delphinidin, delphinidin-3-glucoside, delphinidin-3-galactoside, petunidin, petunidin-3-glucoside, petunidin-3-galactoside, malvidin, malvidin-3-arabinoside, malvidin-3-glucoside, malvidin-3-galactoside, kaempferol, hesperidin, gentiodelphin, platyconin, cinerarin and the like.

Suitable examples of anthocyanins from various plants, include, but are not limited to Acer macrophyllum, Cyanidin derivative, Acer platanoides, Cyanidin 3-(2″,3″-digalloyl-beta-glucopyranose (3%), Cyanidin 3-(2″-galloyl-beta-glucopyranose (37%), Cyanidin 3-beta-glucopyranoside (60%), Acerola, Malpighia marginata, Cyanidin-3-glucoside, Cyanidin-3-glucoside, Ajuga reptans, Cyanidin 3-(di-p-coumaroyl) sophoroside-5-glucoside, Apple, Malus spp, Cyanidin 3-galactoside, Cyanidin 3-galactoside, Cyanidin 3-arabinoside, Cyanidin 3-glucoside, Cyanidin 3arabinoside, Cyanidin 3-xyloside, Cyanidin 3glucoside, Cyanidin 3-xyloside, Apricot, Prunus armeniaca, Cyanidin-3-glucoside, Cyanidin-3glucoside, Artic bramble, Rebus spp, Avocado, Persea spp, Acylated cyanidin 3,5-diglucoside, Cyanidin 3-galactoside, Cyanidin 3-galactoside, Banana, Musa acuminata, M. balbisiana, Barberrv, Berberis spp., Cyanidin-glucoside, Cyanidin-glucoside, Barley, Hordeum vulgare, Cyanidin and cyaniding glycosides, Bean, Pheseolus vulgaris (several cultivars), Cyanidin 3-glucoside, Cyanidin 3-glucoside, Cyanidin 3,5-diglucoside, Begonia semperflorens cvs, Cyanidin derivative, Benibana-cha, Camellia sinensis, Cyanidin 3-O-beta-D galactoside, Cyanidin 3-O-beta-D-galactoside, Bellis perennis, 3 Cyanidin 3-derivatives, Bletilla striata, Acylated cyanidin 3,7,3′-triglucoside derivatives, Bilberry, Vaccinium myrtillus, Artemis/Iprona; Indena, Cyanidin-3-galactoside (22%); Cyanidin-3-galactoside, Cyanidin-3-glucoside (9%), Cyanidin-3glucoside, Black beans, Phaseolus, Cyanidin-3-glucoside (96%), Cyanidin-3glucoside, Blackberry (European and American), Moriferi veri, Rubus caesius, R. Alleghniensis, R. argufus, R. cuneifolius, R. setosus, R. trivials, Cyanidin-glucoside (70-100%), Cyanidin-glucoside, Cyanidin-rutinoside, Black grapes, Many varieties, Black potatoes, Solanumtuberosum tuberosum, Cyanidin-glycosides, Black raspberry, Rubus occidentalis, Cyanidin-sambubloside (20%); Cyanidin-sambubloside, Cyanidin-xylosylrutinoside (40%); Cyanidin-glucoside, Cyanidin-glucoside, (17%), Cyanidin-rutinoside (23%), Black soybeans, Glycine max, Cyanidin-3-glucoside (96%), Cyanidin-3-glucoside, Blueberries, Five common Vaccinium spp, Cyanidin-glucoside (3%); Cyanidin-glucoside, Cyanidin-galactoside (3%), Cyanidin galactoside, Cyanidin-arabinoside (3%), Cyanidin-3-arabinoside, Bog whortleberry, Vaccinium uliginosum, Cyanidin-3-glucoside (14%), Cyanidin 3 glucoside (14%), Cyanidin #arabinoside (10%), Cyanidin-3-arabinoside (110%), Cyanidin 3-galactoside (6.5%), Cyanidin-3-galactoside (6.5%), Boysenberry, new Zealand, Cyanidin-3-sophoroside (44.5%), Cyanidin-3-glucoside, Cyanidin-3-glucoside (26.4%), Cyanidin-3 glycosylrutinoside (25.8%), Cyanidin-rutinoside (3.3%), Buckwheat, Fagopyrum species, Cyanidin-3-glucoside, Cyanidin-3-glucoside, Cyanidin 3-galactoside, Cyanidin-3-galactoside, Cacao, Theobroma cacao, Cyanidin 3-glucoside (suspected), Cyanidin-3-glucoside (suspected), Celery, Apium spp, Cherry laurel, Prunus laurocerasus, Cyanidin-3-arabinoside, Cyanidin-3-arabinoside, Chicory, Cichorium intybus, Cyanidin 3-glucoside, Cyanidin 3-glucoside, Chive, Allium schoenoprasum, Cyanidin-3-glucoside, Cyanidin-3-glucoside, Cyanidin-3-acetylglucoside, Cyanidin 3-(6 malonylglucoside), Cyanidin 3-(3,6 dimalonylglucoside), Chokeberry, Aronia melanocarpa, Artemis/Iprona, Cyanidin-3-galactoside (64.5%), Cyanidin-3-galactoside, Cyanidin-3-arabinoside (28.9%), Cyanidin-3 arabinoside, Cyanidin-3-glucoside (2.4%), Cyanidin-3 glucoside, Cyanidin-3-xyloside (4.2%), Cyanidin-3-xyloside, Coffee, Coffea arabica cv. Bourbon Vermelho, Cyanadin-3-glycoside, Cyanadin 3,5-diglyeoside, Cyanadin 3-glycoside, Cotoneaster, Cotoneaster Medic. Spp, Cyanidin 3-glucoside, Cyanidin 3-glucoside, Cyanidin 3-galactoside, Cyanidin 3-rutinoside, Cyanidin 3 galactoside, Cowberry or Lingonberry, V. vitisidaea, Cyanidin 3-galactoside Cyanidin 3-arabinoside, Cyanidin 3-galactoside, Cyanidin 3-glucoside, Cyanidin 3 arabinoside, Cyanidin 3 glucoside, Chimonanthus praecox, Cyanidin 3-O-glucoside, Cyanidin-3-O-glucoside, Acylated cyanidin 3-0-glucoside, Cyanidin glycoside, Cranberry (American and European), Vaccinium macrocorpon, Ocean Spray, Cyanidin-galactoside (16-24%), Cyanidin-galactoside, V. oxycoccus, Cyanidin-arabinoside (13-25%), Cyanidin arabinoside, CrOwberry, Empetrum nigrum, Cyanidin 3-glucoside Cyanidin 3,5-diglucoside, Cyanidin 3-glucoside, Cyanidin 3-rutinoside, Cyanidin 3-sophoroside, Chrysanthemum, Dendranthema Grandiflorum, Cyanidin 3-O-(6′-O-malonyl-beta-glucopyranoside, Currant (red and black), Ribes rubrum, R. nigrum, Cyanidin-glucoside (2-10%), Cyanidin-glucoside, Cyanidin sambubioside, Cyanidin-rutinoside (8-17%), Cyanidin-sambubioside (9-31%), Cyanidin-sophoroside (4-9%), Cyanidin xylosylrutinoside (28-73%), Cyanidin glucosylrutinoside (14-28%), Cyneinonurn coccineum, Cyanidin 3-O-glucoside (92%), Cyanidin 3-O-glucoside (92%), Cyanadin 3-O-(6-O rhamnosylglucoside (8%), Danewort, Sambucus ebulus, Cyanidin 3-xylosylglucoside, Cyanidin 3-sambubioside, Cyanidin 3 sambubloside, Cyanidin 3-glucoside, Cyanidin 3-sambubioside-5-glucoside, Cyanidin 3,5 diglucoside, Cyanidin 3-glucoside, Cyanidin 3-arabinoglucoside, Dendrobium, Phalaenapsis spp, Cyanidin derivatives, Dwarf dogwood, Comus suecica, Cyanidin 3-glucoside (4%), Cyannidin 3-glucoside (4%), Cyanidin 3-galactoside (16%), 2 Cyanidin derivatives (80%), Echinacea, Echinacea spp., Eldenberry, Sambucus nigra, Artemis/Iprona, Cyanidin-3-glucoside (42%), Cyanidin-3-glucoside, Cyanidin-3-sambubioside (43%) Cyanidin-3,5-diglucoside (2%), Cyanidin-3 sambubloside-5 glucoside (9%), Gentians spp, Cyanidin 3-O-beta-D-glucoside and 3 other derivatives, Cyanidin 3-O-beta-D-glucoside, Fatsia japonica, Cyanidin 3-lathyroside, Feijoa, Feijoa sellowiana, Cyanidin 3-glucoside, Cyanidin 3-glucoside, Fig, Ficus carica spp, Cyanidin 3-rhamnoglucoside, Cyanidin 3,5-diglucoside, Cyanidin 3-glucoside, Forsythia X, intermedia cv, Spring Glory, Cyanidin derivatives, Garlic, Allium sativum, Cyanidin 3-glucoside, Cyanidin 3-glucoside, Cyanidin 3-glucoside, Cyanidin 3-glucoside monoacylated, Cyanidin 3-glucoside triacylated, Ginseng, Panax ginseng, Panax quinquefolius, Cyanidin 3-O-β-D-xylopyranyl-(12)-β-D-glucopyranoside, Globe artichoke, Cynara scolymus, Cyanidin 3-caffeylglucoside, Cyanidin 3-caffeylsophoroside, Cyanidin 3-dicaffeylsophoroside, Gooseberry, Ribes spp, Cyanidin 3-glucoside, Cyanidin 3-glucoside, Cyanidin 3-rutinoside, Grape, Vinis vinifera, Cyanidin 3-monoglucoside, Cyanidin 3-monoglucoside, Cyanidin 3-monoglucoside-acetate, Cyanidin 3-monoglucoside-p-coumarate, Guava, Psidium guajavica, Cyanidin 3-glucoside, Cyanidin 3-glucoside, Hawthorn, Crataegus spp, Cyanidin 3-galactoside, Cyanidin 3-galactoside, Cyanidin 3-arabinoside, Cyanidin 3-glucoside, Cyanidin 3 glucoside, Hibiscus or Roselle, Hibiscus sabdariffa, Cyanidin-sambubioside (30%), Hollyhock, Althaea rosea, Cyanidin 3-glucoside, Cyanidin 3-rutinoside, Cyanidin 3-glucoside, Other cyaniding glucosides, Honeysuckle, Lonicera nitida, Cyanidin 3-rutinoside, Cyanidin 3-glucoside, Cyanidin 3-glucoside, Japanese garden iris, Iris ensata, Cyanidin 3RG, Cyanidin 3RG5G, Cyanidin 3Rgac5G, Ipornoea purpurea, Six acylated cyanidin 3-sophoroside-5 glucosides, Java plum, Mytciana jaboticaba, Cyanidin 3-glucoside, Cyanidin 3-glucoside, Jerusalem artichoke, Helianthus tuberosus, Kokum, Garcinia indica, Cyanidin 3-glucoside, Cyanidin 3-glucoside, Cyanidin 3-sambubioside, Cyanidin 3-sambubioside, Laelioeattleya cv Mini purple, Acylated cyaniding derivatives, Lactuca saliva, Cyanidin 3-O-(6″-malonylglucoside), Loganberry, Rubus loganbaccus, Cyanidin-sophoroside (48.1%), Cyanidin-glucoside, Cyanidin-glucoside (21.6%), Cyanidin-rutinoside (6.2%), Lupine, Lupinus spp, Cyanidin glycosides, presence confirmed, Lychee, Litchi chinensis, Cyanidin 3-glucoside, Cyanidin 3-glucoside, Cyanidin 3-galactoside, Cyanidin 3-rutinoside, Cyanidin 3 galactoside, Maize, Zea mays, Cyanidin 3-glucoside, Cyanidin 3-glucoside, Cyanidin 3-(6″-malonylglucoside) Cyanidin 3(3″,6″dimalonyl-glucoside) Mango, Mangifera indica, (Cyanidin glycosides, Mangosteen, Garcina mangostana, Cyanidin 3-sophoroside, Cyanidin 3-glucoside, Cyanidin 3-glucoside, Maqul, Aristotella chilensis, Cyanidin 3-,5-diglucoside, Matthiola incana, Four acylated cyaniding 3-sambubloside-5 glucosides, Millet, Pernnisetum americanum, Cyanidin 3-glucoside, Cyanidin 3-glucoside, Mountain ash berry, Sorbus spp, Cyanidin 3-galactoside, Cyanidin 3,5-diglucoside Cyanidin 3-β-D glucopyranoside, Mulberry, Morus nigra, Cyanidin 3-glucoside, Cyanidin 3-glucoside, Cyanidin 3,5-diglucoside, Cyanidin 3-rutinoside, Cyanidin 3-sophoroside, Myrtle berry, Myrtus communis, Cyanidin 3-glucosides, Cyanidin 3-glucosides, Cyanidin 3-diglucosides, Olive, Olea europaea, Cyanidin 3-rutinoside, Cyanidin 3-glucoside, Cyanidin 3-glucoside, Cyanidin derivatives, Onion, Allium sepa, Cyanidin 3-glucoside, Cyanidin 3-glucoside, Cyanidin 3-diglucoside, Cyanidin 3-laminarioside, Orange, Citrus sinensis, Cyanidin 3-glucoside (95%), Cyanidin 3-glucoside, Cyanidin 3,5-diglucoside, Passion fruit, Pasiflora edulis, Cyanidin 3-glucoside, Cyanidin 3-glucoside, Pea, Pisum sativurn, Cyanidin 3-sophoroside glucosides, Cyanidin 3-sambubioside-5-glucosides, Peach, Prunus persica, Cyanidin 3-glucoside, Cyanidin 3-glucoside, Cyanidin 3-rutinoside, Cyanidin derivatives, Peanut, Arachis hypogaea, Cyanidin glucosides, Pear, Pyrus communis, Cyanidin 3-galactoside, Cyanidin 3-galactoside, Cyanidin 3-arabinoside, Cyanidin 3-arabinoside, Perilla, Perilla frutescens, Cyanidin 3,5-diglucoside, Cyanidin 3,5-derivatives, Petunia spp, Cyanidin 3-rutinoside, Phalsa, Grewia spp, Cyanidin 3-glucoside, Cyanidin 3-glucoside, Pineapple, Anans comosus, Cyanidin 3-galactoside, Cyanidin 3-galactoside, Pistachio, Pistacia vera, Pragmites australis, Cyanidin-3 derivatives, Plum, 2000 varieties, 15 species, Cyanidin-glucoside (37%), Cyanidin glucoside, Cyanidin-rutinoside (45%), Pomegranate, Punica granatam, Cyanidin-glucoside (30%), Cyanidin-glucoside, Cyanidin-diglucoside (17%), Purple carrot, Daucus carota, Cyanidin-glucoside, Cyanidin-glucoside, Cyanidin-glucosylgalactoside, Cyanidin-galactoside, Cyanidin-digalactoside, Cyanidin-galactoside, Quince, Cydonia oblonga, Cyanidin-3 glucoside, Cyanidin 3,5-diglucoside, Cyanidin derivatives, Radish, Raphanus sativus, Acylated cyanidin 3-sophoroside-5-glucoside, Acylated cyanidin 3 diglucoside-5-glucoside, Red cabbage, Brassica oleracea var capitata, Cyanidin glycosides, Reed, Phalaris arundinacea, Cyanidin 3-glucoside, Cyanidin 3-glucoside, Cyanidin 3-(6″-malonylglucoside), Cyanidin 3 (3″,6″dimalonyl-glucoside), Red onion, Allium cepa, Cyanidin 3-glucoside, Cyanidin 3-glucoside, Acylated cyanidin 3-glucoside derivatives, Red petunia, Petunia spp, Cyanidin 3-glucoside, Cyanidin 3-glucoside, Cyanidin 3-sophoroside, Red raspberry, Rubus idaeus, Cyanidin glucoside (17%), Cyanidin-glucoside, Cyanidin-rutinoside (7%), Cyanidin-sophoroside (50%), Cyanidin glycosylrutinoside (26%), Cyanidin-diglucoside, Rhubarb, Rneum spp, Cyanidin 3-glucoside, Cyanidin 3-glucoside, Cyanidin 3-rutinoside, Rice, Oryza spp, Cyanidin 3-glucoside, Cyanidin 3-glucoside, Cyanidin 3-rhamnoside, Cyanidin 3,5-diglucoside, Rosehip, Rosa canina, Cyanidin 3-rutinoside, Cyanidin 3-glucoside, Cyanidin 3-glucoside, Cyanidin 3,5-diglucoside, Rye, Secale cereale, Cyanidin 3-glucoside, Cyanidin 3-glucoside, Cyanidin 3-rhamnosylglucoside, Cyanidin 3-rhamnosyldiglucoside, Cyanidin 3-rutinoside, Cyanidin 3-rutinoside derivatives, Cyanidin 3-gentiobioside, Sheepberry, Viburnum spp, Cyanidin 3-glucoside, Cyanidin 3-glucoside, Cyanidin 3-arabinosylsambubioside, Sorghum, Sorghum bicolor, Cyanidin, Cyanidin glycosides, Sparkleberry, V. arboreum, Cyanidin 3-glucoside, Cyanidin 3-glucoside, Cyanidin 3-arabinoside, Cyanidin 3-galactoside, Strawberry, Fragaria ananassa, Cyanidin-glucoside (minor), Cyanidin-glucoside, Sunflower, Hellanthus annuus, Cyanidin 3-glucoside, Cyanidin 3-glucoside, Acylated cyanidin 3-glucoside, Cyanidin 3-xyloside, Cyanidin 3-xyloside, Acylated cyanidin 3-xyloside, Cyanidin 3-vanillyl sambubioside, Sweet cherry, Prunus avintn, Cyanidin-glucoside, Cyanidin-glucoside, Cyanidin-rutinoside; Cyanidin 3-suphoroside, Sweet potato, Ipornoea batatas Sophronitis coccinea, Cyanidin derivatives, Five acylated cyanidin 3,3′,7-triglucosides, Tamarillo or tomato tree, Cyphomandrea betacea, Cyanidin 3-rutinoside, Cyanidin 3-glucoside, Cyanidin 3-glucoside, Tamarind, Tamarindus indica, Cyanidin 3-glucoside, Cyanidin 3-glucoside, Taro, Colocasia esculenta, Cyanidin 3-glucoside, Cyanidin 3-glucoside, Cyanidin 3-rutinoside, Tart Cherry (balaton), Prunus cerasus cv. Balaton, Nutrilite, Cyanidin-3-rutinoside-hexose (75%), Cyanidin-3-rutinoside-pentose (3%), Cyanidin-3-rutinoside (18%), Tart cherry (montmorency), Prunus cerasus cv. Montmorency, Nutrilite, Cyanidin-3-sophoroside (80%), Cyanidin-3-glucoside (20%), Cyanidin-3-glucoside (20%), Tulip, Tulipa spp, Cyanidin 3-O-(6″-rhamnosylglucosides), Cyanidin 3-O-derivative, Turnip, brissica rapa, Cyanidin 3-glucoside, Cyanidin 3-glucoside, Cyanidin 3-diglucoside-5-glucoside, Water lily, Nymphasa alba, Cyanidin 3-0-(6″-acetyl-beta-galactopyrosinase (23%), Cyanidin 3-0-galactoside (2%), Cyanidin 3-O-galactoside (2%), Weigela spp, Cyanidin 3-O-glucoside, Cyanidin 3-O-glucoside, Cyanidin 3-O-glucoside xylose, Wheat, Triticum spp, Cyanidin 3-glucoside, Cyanidin 3-glucoside, Acylated cyanidin glucoside, Cyanidin 3-rutinoside, Acylated cyanidin 3-rutinoside, Cyanidin 3-gentiobioside, Wild rice, Zizania aquatica, Cyanidin 3-glucoside, Cyanidin 3-glucoside, Cyanidin 3-rhamnoglucoside, and Yam, Dioscoracea spp, Cyanidin 3,5-diglucoside, Cyanidin 3-glucoside, Cyanidin 3-glucoside, Cyanidin 3-rhamnoglucoside, Cyanidin 3-gentiobioside, Acylated cyanidin glucosides.

The term “anthocyanin” as used herein is intended to refer not only to monomeric anthocyanins, but also refers to dimeric and polymeric (i.e. containing from 3 to 20 anthocyanidin monomer residues) forms of anthocyanins and to leucoanthocyanidins (also known as flavan-3,4-diols). The anthocyanins can comprise substitutions (e.g. alkyl, alkoxy groups etc.) and in particular can be O-glycosylated, as described above.

The anthocyanin in the composition can be a single anthocyanin, or comprise a mixture of anthocyanins. In particular, the anthocyanin is selected from the group consisting of: malvidin, cyanidin, delphinidin, paeonidin, pelargonidin and petunidin, and glycosides thereof A typical example is malvin (malvidin diglucoside) chloride, which is commercially available in a purified form. Alternatively the anthocyanin can be obtained by extracting anthocyanin containing plants such as grape, black carrot, red cabbage, blackberry, blackcurrent, cranberry and the like as described above.

The phrase “stabilized carotenoid composition” as used herein is intended to mean that the carotenoid, either as a glycoside (esterified) or free form (de-esterified), for example, at about 37° C., pH=about 7.0, remains at least about 50% undegraded from the original percentage of carotenoid for at least about 3.5 hours. Likewise, the phrase includes pH values of about 4, about 5, about 6 and about 8 with similar stability.

The term “stabilizing compound” as used herein is intended to include those compounds that have at least one —SH group. Not to be limited by theory, it is believed that an interaction occurs between the thiol group and the anthocyanin such that the thiol containing group is oxidized (often to a disulfide, —S—S—) and the anthocyanin receives a hydrogen atom, which is then later liberated.

Suitable examples of stabilizing compounds include beer yeast, dihydrolipoic acid, salts of dihydrolipoic acid such as amino acid salts, cysteine, derivatives of cysteine, such as N-acetylcysteine, glutathione, salts of glutathione, SH-proteinase such as papain, bromelain, ficin, ehymopapain and mixtures thereof, SH-metalloproteinases, peptides containing cysteine, peptides containing glutathioine, fermented oyster extract, fermented bean curd, thiolated chitosans, thiolated gelatins or mixtures thereof.

In one aspect, the mole ratio of stabilizing compound to anthocyanin is between about 0.1 to about 10, more particularly between about 0.5 to about 5 and even more particularly about 1 to about 1.

The term alpha lipoic acid is intended to mean the compound represented by the formula:

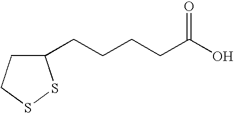

as one of its enantiomers or racemic form.

Alpha-lipoic acid was first isolated as an acetate replacing factor. It is slightly soluble in water and soluble in certain organic solvents. Alpha-lipoic acid was initially identified as a vitamin after its isolation, but it was later found to be synthesized by mammals, including humans, as well as by plants. The complete enzyme pathway that is responsible for the de novo synthesis has not yet been definitively elucidated. Several studies have indicated that octanoate serves as the immediate precursor for the 8-carbon fatty acid chain, and cysteine appears to be the source of sulfur. As an amide (lipoamide), it functions as a cofactor in the multienzyme complexes that catalyze the oxidative decarboxylation of alpha-keto acids such as pyruvate, alpha-keto glutarate, and branched chain alpha-keto acids.

Alpha-lipoic acid is one of the strongest naturally occurring antioxidants. Alpha-lipoic acid (LA) is also known as thioctic acid, 1,2-dithiolane-3-pentanoic acid, 1,2-dithiolane-3-valeric acid and 6,8-thioctic acid. Alpha-lipoic acid has a chiral carbon atom and occurs in two enantiomeric forms (R- and S-). The form of alpha-lipoic acid sold in stores is a synthetic mixture of the natural isomer (R-) and the unnatural isomer (S-). The natural form of R-LA is not as stable as the synthetic mixture. One manufacturer, Asta Medica, sells R-LA for diabetes and has made a stable form of R-LA by crystallizing it with Tris buffer, a commonly used synthetic, but unnatural, buffer.

Various enantiomeric forms of alpha-LA, and combinations and derivatives thereof (including its reduced form), have been used to treat numerous conditions. For example, LA's have been used in the treatment of circulatory disorders. LAs and vitamins have been found useful for producing analgesic, anti-inflammatory, antinecrotic, anti-diabetic and other therapeutic effects. Certain alkylated derivatives of LA have been used in treatment of retroviral diseases.

Alpha-lipoic acid, and its reduced form, dihydrolipoic acid (DHLA) have antioxidant properties. Lipoate (a term for carboxylic acid esters and salts), or its reduced form, DHLA, reacts with reactive oxygen species such as superoxide radicals, hydroxyl radicals, hypochlorous acid, peroxyl radicals, and singlet oxygen. It also protects membranes by interacting with vitamin C and glutathione, which may in turn recycle vitamin E. In addition to its antioxidant activities, DHLA may exert prooxidant actions to reduction of iron. Alpha-lipoic acid administration has been shown to be beneficial in a number of oxidative stress models such as ischemia-reperfusion injury (IRI), diabetes (both alpha-lipoic acid and DHLA exhibit hydrophobic binding to proteins such as albumin, which can prevent glycation reactions), cataract formation, HIV activation, neurodegeneration, and radiation injury. Furthermore, lipoate can function as a redox regulator of proteins such as myoglobin, prolactin, thioredoxin, and NF-kappa-B transcription factor.

Lipoates may also have other activities. For example, DHLA has been found in vitro to be an anti-inflammatory agent, which at the same time interferes with nitric oxide release from inflammatory macrophages and protects target cells from oxygen radical attack.

Lipoic acid is also a coenzyme for several enzymes. Lipoic acid is a coenzyme for both alpha-keto acid dehydrogenase complex enzymes (i.e. pyruvate dehydrogenase complex and alpha-keto glutarate dehydrogenase complex), branched chain alpha-keto acid dehydrogenase complex, and the glycine cleavage system. In the enzyme system, the body forms a multi-enzyme complex involving lipoic acid, that breaks down molecules of pyruvate produced in earlier metabolism, to form slightly smaller, high energy molecules, called acetyl-coenzyme A. This results in molecules that can enter into a series of reactions called the citric acid cycle, or Krebs cycle, which finishes the conversion of food into energy. Essentially, lipoic acid stimulates basal glucose transport and has a positive effect on insulin stimulated glucose uptake.

Under physiological conditions, LA exists as lipoamide in at least five proteins where it is covalently linked to a lysyl residue. Four of these proteins are alpha-ketoacid dehydrogenase complexes, the pyruvate dehydrogenase complex, the branched chain keto-acid dehydrogenase complex and the alpha-ketoglutarate dehydrogenase complex. Three lipoamide-containing proteins are present in the E2 enzyme dihydrolipoyl acyltransferase, which is different in each of the complexes and specific for the substrate of the complex. One lipoyl residue is found in protein X, which is the same in each complex. The fifth lipoamide residue is present in the glycine cleavage system.

Recently LA has been detected in the form of lipoyllysine in various natural sources. In the plant material studied, lipoyllysine content was highest in spinach. When expressed as weight per dry weight of lyophilized vegetables, the abundance of naturally existing lipoate in spinach was over three- and five-fold higher than that in broccoli and tomatoes, respectively. Lower concentrations of lipoyllysine were also detected in garden pea, Brussels sprouts and rice bran.

In animal tissues, the abundance of lipoyllysine in bovine acetone powders can be represented in the following order of 1) kidney, 2) heart, 3) liver, 4) spleen, 5) brain, 6) pancreas and 7) lung.

α-lipoic acid is known as an active pharmaceutical agent for treating various diseases, such as liver diseases or diabetic and alcoholic polyneuropathy. DE 198 18 563 discloses the use of α-lipoic acid or its salts for reducing appetite and/or reducing body weight. Therefore, lipoic acid (in combination with the xanthophyll) provides a nutritional benefit to provide treatment to such diseases.

Dihydrolipoic acid is the reduced (has electrons added) form of lipoic acid (thioctic acid). When DHLA is oxidized (has electrons removed) lipoic acid is produced. It should be understood that DHLA can be either the R or S enantiomer or it can be racemic. Likewise, lipoic acid can also be enantiomerically pure or racemic.

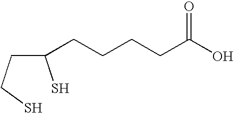

Dihydrolipoic Acid (Dihydrothioctic Acid)

The term “aprotic solvent” is intended to include those solvents that do not include an acidic proton, a hydroxyl proton or easily hydrolysable hydrogen atom or a solvent that does not yield or accept a proton. Suitable aprotic solvents include, for example, methylene chloride, C5 to C10 alkanes (branched and unbranched), aromatic hydrogens, etc.

The present invention also pertains to methods of preparing the stabilized compositions described herein.

The present invention provides that the stabilizing compound(s) can optionally be admixed with the anthocyanin containing plant material and carotenoid during the extraction and/or manufacturing process, thereby reducing or eliminating the oxidative destruction of the anthocyanin that commonly occurs upon processing and even upon storage. For example one or more of the stabilizing compounds in the ratios generally described herein can be added into the extraction medium (solvent) during the extraction process as disclosed in US Patent Publication 2002/0018821, published Feb. 14, 2002 by Chrystele Soulier et al., the contents of which are incorporated herein in their entirety.

Typically, the anthocyanin extract is mixed directly with the stabilizing compound. This can be accomplished by simply mixing, grinding, combining, etc. the two materials as solids or by dissolution in a solvent, such as water. Additional additives, such as carriers, vitamins, antioxidants, etc., as described herein below, can be added to the mixture by conventional methods. This mixture can then be combined with the carotenoid. Optionally, the mixture does not include the stabilizing compound and is only a mixture of the anthocyanin and carotenoid. Optionally, the mixture does not include the anthocyanin and is only a mixture of the stabilizing compound and the carotenoid.

In one embodiment, a red fruit extract containing anthocyanosides, is taken up in an aqueous solution and optionally treated with an —SH stabilizing compound as described herein. The aqueous extract is cooled until it reaches a homogeneous temperature of less than 15° C. The aqueous extract is filtered and is optionally treated with an —SH stabilizing compound as described herein, the permeate obtained is recovered and loaded onto a macrocrosslinked polymeric resin. The resin is then rinsed with demineralized water, optionally treated with an —SH stabilizing compound as described herein and then the resin obtained is eluted with an alcoholic eluting solution, which may optionally be treated with an —SH stabilizing compound as described herein. The eluate obtained is concentrated, optionally treated with an —SH stabilizing compound as described herein and then dried. The carotenoid can be added during any point of the described process.

In another embodiment, the process of stabilization is carried out on an alcoholic red fruit extract obtained according to the following process. The pulp is first separated from the whole red fruit and the pulp is then brought into contact with an alcoholic extraction solution which can optionally contain an —SH stabilizing compound as described herein. The solid phase is separated from the liquid phase and the liquid phase can be optionally treated with an —SH stabilizing compound as described herein. The major portion of the residual alcohol contained in the liquid phase is evaporated under vacuum so as to obtain an alcoholic concentrate. The carotenoid can be added anytime during the process.

Advantageously, the solvent used for the alcoholic extraction is chosen from the group comprising methanol, ethanol, butanol and acetone.

In practice, the alcoholic extraction of anthocyanin is carried out at room temperature in at least two successive steps, each lasting 20 minutes. The solvent is then evaporated off. In addition, it is also possible to carry out the extraction of the anthocyanosides not from the pulp alone, but from whole fruits.

According to the invention, the process of stabilization can be carried out starting with extracts of fruits which are commercially available or with prepurified anthocyanoside extract, each provided in liquid or powdered form. In this case, the fruit extract or the prepurified extract can then be taken up, before the purification step, either with alcohol, in particular methanol, or with water and treated with an —SH stabilizing compound as described herein. The material is then combined with one or more carotenoids.

In the process of purification of the invention, the cooling of the fruit extract is advantageously carried out until the temperature of the extract is homogeneous and less than 10° C., in particular, less than 5° C., with the temperature being maintained for at least about twelve hours.

With regard to the step of filtration of the aqueous extract or alcoholic extract, it may be carried out on a cellulose filter or a stainless steel gauze with a cut-off of between 0 and 100 micrometers or equivalent.

In order to further increase the titer and the concentration of anthocyanosides in the final extract, the alcoholic solution with which the anthocyanosides are eluted from the resin is an aqueous solution of ethanol whose ethanol concentration is between 10 and 90%, advantageously close to 40%.

The eluate obtained is concentrated at a controlled temperature in the region of 30° C. and then freeze-dried or spray-dried so as to obtain a powder.

The ratio of an anthocyanin to a —SH containing compound to a carotenoid is from about 1 to about 500 (anthocyanin):about 0.1 to about 50 (—SH):to about 1 (carotenoid) (w/w/w), more particularly from about 30 to about 20; about 3 to about 2; about 1; and more particularly from about 80:about 9:about 3, all w/w/w. In particular, a bilberry extract can contain 36% by weight anthocyanosides.

In one embodiment, for example, a concentrate of bilberry extract, L-cysteine hydrochloride and lutein are combined (9:1:1, w/w/w) and spray dried to afford a stabilized bilberry extract as a powder. In general, the bilberry extract to free cysteine to lutein ratio is approximately 10:1:1, w/w/w.

The present invention further pertains to methods of treatment of various ailments by administration of a therapeutically effective amount of the compositions described herein. Ailments include, the need for increased antioxidant capacity, arthrosclerosis, reduction of pain, inflammation. Reduction or the elimination of pain includes various forms of pain including arthritis, dysmenorrheal, headaches, joint pain, muscular pain, osteoarthritis, age-related macular degeneration (AMD), cataracts, retinopathy, and combinations thereof.

Therefore, the present invention further provides bioavailable stabilized anthocyanin/carotenoid (including either anthocyanin/carotenoid, a compound having at least one —SH group and a carotenoid, or a combination of an anthocyanin/a compound having at least one —SH group and a carotenoid) compositions that are useful to treat various afflictions noted herein. The compositions of the invention can be administered by a number of methods, as discussed infra.

The compositions according to the present invention are especially useful as food ingredients, in the cosmetic industry and/or in a pharmaceutical product. Especially the antioxidative properties of lutein and its positive effects on the skin and the eyes are fully preserved in the compound according to the present invention.

The compositions of the invention can be incorporated into various foods, drinks, snacks, etc. In one aspect, the composition can be sprinkled onto a food product, prior to consumption. If sprinkled onto a food product, a suitable carrier such as starch, sucrose or lactose, can be used to help distribute the concentration of the xanthophyll(s) making it easier to apply to the food product.

The compositions of the present invention can also be provided as supplements in various prepared food products. For the purposes of this application, prepared food product means any natural, processed, diet or non-diet food product to which a composition of the invention has been added. The compositions of the present invention can be directly incorporated into many prepared diet food products, including, but not limited to diet drinks, diet bars and prepared frozen meals. Furthermore, the compositions of the inventions can be incorporated into many prepared non-diet products, including, but not limited to candy, snack products such as chips, prepared meat products, milk, cheese, yoghurt, sport bars, sport drinks, mayonnaise, salad dressing, bread and any other fat or oil containing foods. As used herein, the term “food product” refers to any substance fit for human or animal consumption.

The compositions of the invention can be added to various drinks, such as fruit juices, milkshakes, milk, etc.

The preferred method of administration is oral. The compositions of the invention can be formulated with suitable carriers such as starch, sucrose or lactose in tablets, capsules, solutions, syrups and emulsions. The tablet or capsule of the present invention can be coated with an enteric coating that dissolves at a pH of about 6.0 to 7.0. A suitable enteric coating, which dissolves in the small intestine but not in the stomach, is cellulose acetate phthalate.

Formulation of the compositions of the invention into a soft gel capsule can be accomplished by many methods known in the art. Often the formulation will include an acceptable carrier, such as an oil, or other suspending or emulsifying agent.

Suitable optional carriers include but are not limited to, for example, fatty acids, esters and salts thereof, that can be derived from any source, including, without limitation, natural or synthetic oils, fats, waxes or combinations thereof. Moreover, the fatty acids can be derived, without limitation, from non-hydrogenated oils, partially hydrogenated oils, fully hydrogenated oils or combinations thereof. Non-limiting exemplary sources of fatty acids (their esters and salts) include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, wheat germ oil, tallow, beef tallow, butter, chicken fat, lard, dairy butterfat, shea butter or combinations thereof.

Specific non-limiting exemplary fish or marine oil sources include shellfish oil, tuna oil, mackerel oil, salmon oil, menhaden, anchovy, herring, trout, sardines or combinations thereof. In particular, the source of the fatty acids is fish or marine oil (DHA or EPA), soybean oil or flaxseed oil. Alternatively or in combination with one of the above identified carrier, beeswax can be used as a suitable carrier, as well as suspending agents such as silica (silicon dioxide).

The formulations of the invention are also considered to be nutraceuticals. The term “nutraceutical” is recognized in the art and is intended to describe specific chemical compounds found in foods that can prevent disease or ameliorate an undesirable condition.

The formulations of the invention can further include various ingredients to help stabilize, or help promote the bioavailability of the components of the beneficial compositions of the invention or serve as additional nutrients to an individual's diet. Suitable additives can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals can be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Various additives can be incorporated into the present compositions. Optional additives of the present composition include, without limitation, hyaluronic acid, phospholipids, starches, sugars, fats, antioxidants, amino acids, proteins, flavorings, coloring agents, hydrolyzed starch(es) and derivatives thereof or combinations thereof.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene; natural components such as camosol, camosic acid and rosmanol found in rosemary and hawthorn extract, proanthocyanidins such as those found in grapeseed or pine bark extract, and green tea extract.

Compositions comprising the xanthophyll of the invention can be manufactured by methods of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries that facilitate processing of the xanthophyll compositions into preparations that can be used.

The compositions of the invention can take a form suitable for virtually any mode of administration, including, for example, oral, buccal, systemic, injection, transdermal, rectal, vaginal, etc., or a form suitable for administration by inhalation or insufflation.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the xanthophyll extract compositions in aqueous or oily vehicles. The compositions can also contain formulating agents, such as suspending, stabilizing and/or dispersing agent. The formulations for injection can be presented in unit dosage form, e.g., in ampoules or in multidose containers, and can contain added preservatives.

Alternatively, the injectable formulation can be provided in powder form for reconstitution with a suitable vehicle, including but not limited to sterile pyrogen free water, buffer, dextrose solution, etc., before use. To this end, the xanthophyll compositions can be dried by any art-known technique, such as lyophilization, and reconstituted prior to use.

For transmucosal administration, penetrants appropriate to the barrier to be permeated are used in the formulation. Such penetrants are known in the art.

For oral administration, the compositions of the invention can take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art with, for example, sugars, films or enteric coatings.

Liquid preparations for oral administration can take the form of, for example, elixirs, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol, or fractionated vegetable oils); and preservatives (e.g., methyl or propyl p hydroxybenzoates or sorbic acid). The preparations can also contain buffer salts, preservatives, flavoring, coloring and sweetening agents as appropriate.

Preparations for oral administration can be suitably formulated to give controlled release of the xanthophyll composition as is well known.

For buccal administration, the compositions can take the form of tablets or lozenges formulated in conventional manner.

For rectal and vaginal routes of administration, the xanthophyll compositions can be formulated as solutions (for retention enemas) suppositories or ointments containing conventional suppository bases such as cocoa butter or other glycerides.

For nasal administration or administration by inhalation or insufflation, the xanthophyll compositions can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound and a suitable powder base such as lactose or starch.

For prolonged delivery, the xanthophyll compositions can be formulated as a depot preparation for administration by implantation or intramuscular injection. The xanthophyll compositions can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch, which slowly releases the xanthophyll compositions for percutaneous absorption, can be used. To this end, permeation enhancers can be used to facilitate transdermal penetration of the compositions. Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other delivery systems can be employed. Liposomes and emulsions are well-known examples of delivery vehicles that can be used to deliver xanthophyll compositions. Certain organic solvents such as dimethylsulfoxide (DMSO) can also be employed, although usually at the cost of greater toxicity.

The compositions can, if desired, be presented in a pack or dispenser device, which can contain one or more unit dosage forms containing the xanthophyll compositions. The pack can, for example, comprise metal or plastic foil, such as a blister pack. The pack or dispenser device can be accompanied by instructions for administration.

Soft gel or soft gelatin capsules can be prepared, for example, without limitation, by dispersing the formulation in an appropriate vehicle (e.g., rice bran oil, and/or beeswax) to form a high viscosity mixture. This mixture is then encapsulated with a gelatin based film using technology and machinery known to those in the soft gel industry. The capsules so formed are then dried to constant weight. Typically, the weight of the capsule is between about 100 to about 2500 milligrams and in particular weigh between about 1500 and about 1900 milligrams, and more specifically can weigh between about 1500 and about 2000 milligrams.

For example, when preparing soft gelatin shells, the shell can include between about 20 to 70 percent gelatin, generally a plasticizer and about 5 to about 60% by weight sorbitol. The filling of the soft gelatin capsule is liquid (principally a carrier such as rice bran oil or wheat germ oil and/or beeswax if desired) and can include, apart from the xanthophyll, a hydrophilic matrix. The hydrophilic matrix, if present, is a polyethylene glycol having an average molecular weight of from about 200 to 1000. Further ingredients are optionally thickening agents and/or emulsifying agent(s). In one embodiment, the hydrophilic matrix includes polyethylene glycol having an average molecular weight of from about 200 to 1000, 5 to 15% glycerol, and 5 to 15% by weight of water. The polyethylene glycol can also be mixed with propylene glycol and/or propylene carbonate.

In another embodiment, the soft gel capsule is prepared from gelatin, glycerine, water and various additives. Typically, the percentage (by weight) of the gelatin is between about 30 and about 50 weight percent, in particular between about 35 and about weight percent and more specifically about 42 weight percent. The formulation includes between about 15 and about 25 weight percent glycerine, more particularly between about 17 and about 23 weight percent and more specifically about 20 weight percent glycerine.

The remaining portion of the capsule is typically water. The amount varies from between about 25 weigh percent and about 40 weight percent, more particularly between about 30 and about 35 weight percent, and more specifically about 35 weight percent. The remainder of the capsule can vary, generally, between about 2 and about 10 weight percent composed of a flavoring agent(s), sugar, coloring agent(s), etc. or combination thereof. After the capsule is processed, the water content of the final capsule is often between about 5 and about 10 weight percent, more particularly 7 and about 12 weight percent, and more specifically between about 9 and about 10 weight percent.

As for the manufacturing, it is contemplated that standard soft shell gelatin capsule manufacturing techniques can be used to prepare the soft-shell product. Examples of useful manufacturing techniques are the plate process, the rotary die process pioneered by R. P. Scherer, the process using the Norton capsule machine, and the Accogel machine and process developed by Lederle. Each of these processes is mature technologies and is all widely available to any one wishing to prepare soft gelatin capsules.

Emulsifying agents can be used to help solubilize the ingredients within the soft gelatin capsule. Specific examples of the surfactant, emulsifier, or effervescent agent include D-sorbitol, ethanol, carrageenan, carboxyvinyl polymer, carmellose sodium, guar gum, glycerol, glycerol fatty acid ester, cholesterol, white beeswax, dioctyl sodium sulfosuccinate, sucrose fatty acid ester, stearyl alcohol, stearic acid, polyoxyl 40 stearate, sorbitan sesquioleate, cetanol, gelatin, sorbitan fatty acid ester, talc, sorbitan trioleate, paraffin, potato starch, hydroxypropyl cellulose, propylene glycol, propylene glycol fatty acid ester, pectin, polyoxyethylene (105) polyoxypropylene (5) glycol, polyoxyethylene (160) polyoxypropylene (30) glycol, polyoxyethylene hydrogenated castor oil, polyoxyethylene hydrogenated castor oil 40, polyoxyethylene hydrogenated castor oil 60, polyoxyl 35 castor oil, polysorbate 20, polysorbate 60, polysorbate 80, macrogol 400, octyldodecyl myristate, methyl cellulose, sorbitan monooleate, glycerol monostearate, sorbitan monopalmitate, sorbitan monolaurate, lauryl dimethylamine oxide solution, sodium lauryl sulfate, lauromacrogol, dry sodium carbonate, tartaric acid, sodium hydroxide, purified soybean lecithin, soybean lecithin, potassium carbonate, sodium hydrogen carbonate, medium-chain triglyceride, citric anhydride, cotton seed oil-soybean oil mixture, and liquid paraffin.

The present invention also provides packaged formulations of the compositions of the invention and instructions for use of the product for appropriate condition(s). Typically, the packaged formulation, in whatever form, is administered to an individual in need thereof. Typically, the dosage requirement is between about 1 to about 4 dosages a day.

Although the present invention describes the preparation, use, manufacture and packaging of the compositions of the invention in soft gelatin capsules for treatment of various conditions, it should not be considered limited to only soft gelatin capsules. Ingestible compositions of the invention can be delivered in traditional tablets, pills, lozenges, elixirs, emulsions, hard capsules, liquids, suspensions, etc. as described above.

The xanthophyll compositions of the invention, or compositions thereof, will generally be used in an amount effective to achieve the intended result, for example in an amount effective to treat or prevent the particular related condition being treated. The composition can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient can still be afflicted with the underlying disorder. For example, administration of a composition of the invention to a patient suffering from pain provides therapeutic benefit not only when the underlying condition is eradicated or ameliorated, but also when the patient reports a decrease in the severity or duration of the physical discomfort associated with the pain.

For prophylactic administration, the composition can be administered to a patient at risk of developing one of the previously described conditions.

The amount of composition administered will depend upon a variety of factors, including, for example, the particular indication being treated, the mode of administration, whether the desired benefit is prophylactic or therapeutic, the severity of the indication being treated and the age and weight of the patient, etc. Determination of an effective dosage is well within the capabilities of those skilled in the art.

Total dosage amounts of a xanthophyll ester derivatized with at least one nutritionally beneficial carboxylic acid residue composition will typically be in the range of from about 0.0001 or 0.001 or 0.01 mg/kg/day to about 100 mg/kg/day, but may be higher or lower, depending upon, among other factors, the activity of the components, its bioavailability, the mode of administration and various factors discussed above. Dosage amount and interval can be adjusted individually to provide plasma levels of the compound(s) which are sufficient to maintain therapeutic or prophylactic effect. For example, the compounds can be administered once per week, several times per week (e.g., every other day), once per day or multiple times per day, depending upon, among other things, the mode of administration, the specific indication being treated and the judgment of the prescribing physician. Skilled artisans will be able to optimize effective local dosages without undue experimentation.

The following paragraphs enumerated consecutively from 1 through 24 provide for various aspects of the present invention. In one embodiment, in a first paragraph (1), the present invention provides a composition comprising a carotenoid and a compounds with at least one —SH group.

2. The composition according to paragraph 1, wherein the composition further comprises an anthocyanin.

3. The composition according to paragraph 1 or 2, wherein the carotenoid is of free form.

4. The composition according to any of paragraphs 1 through 3, wherein the compound with —SH group is L-cysteine or glutathione.

5. The composition according to any of paragraphs 1 through 4, wherein the carotenoid is a xanthophyll or a derivative.

6. The composition according to paragraph 5, wherein the xanthophyll is lutein, zeaxanthin, capsorubin, capsanthin, astaxanthin, canthaxanthin or mixtures thereof.

7. A method to increase the bioavailability of a carotenoid, comprising the step of combining a compound having at least one —SH group and a carotenoid, such that the bioavailability of the carotenoid is increased relative to a composition without the compound having at least one —SH group.

8. The method of paragraph 7, wherein the bioavailability of the carotenoid is increased at least about fifteen percent of the amount of bioavailable carotenoid without the compound having at least one —SH group.

9. The method of paragraph 7, wherein the bioavailability of the carotenoid is increased at least about fifteen percent to about 4 times of the amount of bioavailable carotenoid without the compound having at least one —SH group.

10. A method to increase the bioavailability of a carotenoid, comprising the step of combining an anthocyanin and a carotenoid, such that the bioavailability of the carotenoid is increased relative to a composition without the anthocyanin.

11. The method of paragraph 10, wherein the bioavailability of the carotenoid is increased at least about fifteen percent of the amount of bioavailable carotenoid without the anthocyanin.

12. The method of paragraph 10, wherein the bioavailability of the carotenoid is increased at least about fifteen percent to about 4 times of the amount of bioavailable carotenoid without the anthocyanin.

13. A method to increase the bioavailability of a carotenoid, comprising the step of combining an anthocyanin, a compound having at least one —SH group and a carotenoid, such that the bioavailability of the carotenoid is increased relative to a composition without the anthocyanin and compound having at least one —SH group.

14. The method of paragraph 13, wherein the bioavailability of the carotenoid is increased at least about fifteen percent of the amount of bioavailable carotenoid without the anthocyanin and compound having at least one —SH group.

15. The method of paragraph 13, wherein the bioavailability of the carotenoid is increased at least about fifteen percent to about 4 times of the amount of bioavailable carotenoid without the anthocyanin and compound having at least one —SH group.

16. The method according to any of paragraphs 7 through 15, wherein the carotenoid is of free form.

17. The method according to any of paragraphs 7 through 15, wherein the carotenoid is lutein, zeaxanthin, capsorubin, capsanthin, astaxanthin, canthaxanthin or mixtures thereof.

18. The method according to paragraph 16, wherein the carotenoid is lutein, zeaxanthin, capsorubin, capsanthin, astaxanthin, canthaxanthin or mixtures thereof.

19. The composition or method of any of paragraphs 1 through 18, wherein the anthocyanin, if present, is/are the anthocyanins present in bilberry extract.

20. A composition comprising a carotenoid and an anthocyanin, wherein the anthocyanin is present in an amount sufficient to increase the bioavailability of the carotenoid at least about fifteen percent of the amount of bioavailable carotenoid without the anthocyanin.

21. The composition according to paragraph 20, wherein the carotenoid is of free form.

22. The composition according to either of paragraphs 20 or 21, wherein the carotenoid is a xanthophyll or a derivative.

23. The composition according to paragraph 22, wherein the xanthophyll is lutein, zeaxanthin, capsorubin, capsanthin, astaxanthin, canthaxanthin or mixtures thereof.

24. The composition of any of paragraph 20 through 32, wherein the anthocyanin is a bilberry extract or a black currant extract.

The following examples are not to be meant as limiting but are presented to provide additional information and support for the invention.

### EXAMPLES

### Increased Bioavailability of Lutein

### 1. PROJECT INTRODUCTION

Lutein is an excellent antioxidant, but it degrades very quickly, as a result, the absorption and bioavailability of lutein is very poor. It was discovered herein that anthocyanoids and L-cysteine are powerful antioxidants in aqueous systems, so that increased bioavailability of lutein and related carotenoids are possible.

There are two series of experiments; one is in vitro stability experiment, the other is in vitro cell uptake experiments

### 2. IN VITRO STABILITY EXPERIMENTS

Experiments

Bilberry extract: Omya-Peralta GmbH (Lot No.: BB0823-1)

Black currant extract: Omya-Peralta GmbH (Lot No.: BC6006)

10.0 mg (0.013 mmol) lutein in 10 ml was added to a volumetric flask and diluted to volume with THF. In a small beaker, 0.50 g PEO (20) sorbitan monooleate was dissolved in 15.0 g de-ionized water. 1.0 ml lutein THF solution was added into the water solution and sonicated until a clear yellow micro-emulsion was formed.

10.0 mg (0.006 mmol) bilberry extract was added to a second 10 ml volumetric flask and filled to volume with de-ionized water. 1.0 ml bilberry extract solution was transferred to a small beaker and sonicated until a homogeneous solution was obtained. 1.0 ml of the bilberry solution was placed into a 10 ml colorless volumetric flask and filled to volume with sodium phosphate buffer (pH=7.0). The buffered solution was placed into a 37° C. water bath for 5 hours. UV-VIS spectrometery was used to analyze the degradation ratio. All samples were made alike. Similarly, L-cysteine was used in place of bilberry extract.

2.1 Effect of L-Cysteine Dosage

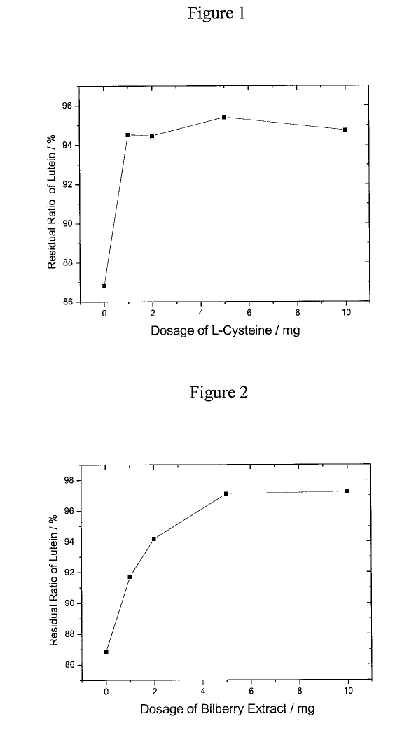

Under neutral environmental conditions, the L-Cysteine had remarkable protection to lutein. Even when L-Cysteine concentration was very low, the effect was pronounced. See FIG. 1 also.

TABLE 1
Effect of L-Cysteine Dosage

| Sample | | |
|---|---|---|
| No. | Sample Name | Residual Ratio/% |
| 1 | Blank Sample* | 86.82 |
| 2 | 1.0 mg (0.8 μmol/ml) L-Cys | 94.52 |
| 3 | 2.0 mg (1.7 μmol/ml) L-Cys | 94.47 |
| 4 | 5.0 mg (4.1 μmol/ml) L-Cys | 95.41 |
| 5 | 10.0 mg (8.2 μmol/ml) L-Cys | 94.74 |

*All sample contain 4.3 μg (0.008 μmol) free lutein

2.2 Effect of Bilberry Extract Dosage

The bilberry extract also protected lutein under neutral environmental conditions. See FIG. 2 also.

TABLE 2
Effect of Bilberry Extract Dosage

| Sample | | |
|---|---|---|
| No. | Sample Name | Residual Ratio/% |
| 1 | Blank Sample | 86.82 |
| 2 | 1.0 mg (0.06 μmol/ml) Bilberry | 91.73 |
| 3 | 2.0 mg (0.12 μmol/ml) Bilberry | 94.18 |
| 4 | 5.0 mg (0.30 μmol/ml) Bilberry | 97.11 |
| 5 | 10.0 mg (0.60 μmol/ml) Bilberry | 97.23 |

2.3 Effect of Bilberry Extract & L-Cysteine Mixture

When bilberry extract and L-Cysteine were combined, the result was better than bilberry extract or L-Cysteine alone.

TABLE 3
Effect of Bilberry Extract & L-Cysteine Mixture

| Sample | | |
|---|---|---|
| No. | Sample Name | Residual Ratio/% |
| 1 | Blank Sample | 86.82 |
| 2 | 1.0 mg (0.06 μmol/ml) Bilberry | 91.73 |
| 3 | 0.2 mg (0.17 μmol/ml)L-Cys | 90.22 |
| 4 | 1.0 mg Bilberry & 0.2 mg L-Cys | 93.49 |

### 3. IN VITRO CELL UPTAKE EXPERIMENT

Experiments

Culturing of CaCo-2 Cells

CaCo-2 cells were cultured in Dulbeccos's Modified Eagle Medium containing 20% fetal bovine serum, 1.2% nonessential amino acids, 0.83 mM L-glutamine, 1.2% penicillin-streptomycin and 0.1% mercaptoethanole in an atmosphere of 5% CO2 and 95% air at 37° C.

Cells were grown in 75 cm² culture-flasks (T75) and sub-cultured after one week (every other day washed with PBS buffer, removed with trypsin and transferred to a new culture flask).

CaCo-2 Test

For experiments, cells were seeded in 6 well plates at a density of 3×10⁵ cells per well and grown in an atmosphere of 5% CO₂ and 95% air at 37° C. 7 to 8 days until confluence was reached. The cells were washed with PBS buffer, incubated with 4 ml medium containing the suspended samples for 30, 60 or 120 minutes.

After the corresponding incubation time, the cells were washed with PBS buffer and removed using 1 ml of TBME. Cells were sonicated 3 times for 30 seconds, centrifuged for 10 min and the pellets were discarded. The supernatant was used as sample for HPLC (lutein). For luteindipalmitinic-ester the supernatant was hydrolyzed by reflux heating in ethanol/HCl.

Pharmacokinetics

Cmax was taken directly from the concentration/time data obtained from the analysis.

The concentration/time data obtained from the analysis were further submitted to non-compartmental calculation of the AUC₀₋₁₂₀ₘᵢₙ using the trapezoidal rule.

Analysis of Lutein

| | |
|---|---|
| Pump: | Merck Quaternary Gradient pump 6200, Merck |
| | AS 2000, HP-MVD 1050, column oven Tech Lab |
| Column: | YMC C30, 300 × 4.6 mm |
| Mobile phase: | TBME/Methanol = 30/70 (v/v) |
| Flow Rate: | 1 mL/min |
| Temperature: | Ambient |
| Injection volume: | 20 μl |
| Detection: | 450 nm |

Standard Preparation (Lutein)

Transfer an appropriate amount of standard into a 10 mL flask and dissolve in 1 mL chloroform. Then fill up to the mark with mobile phase. Dilutions were prepared with the mobile phase.

Data Evaluation

Quantification was performed by external standardization after linear regression analysis.

3.1 Cellular Uptake of Lutein (Free Form) in CaCo-2 Cells

CaCo-2 cells were incubated with 10 mg (0.013 mmol) Lutein/100 mL medium, 10 mg Lutein plus 160 mg bilberry extract (25% anthocyanins, 0.08 mmol)/100 mL medium or 10 mg Lutein plus 180 mg of a mixture of bilberry extract/L-cysteine [equivalent to 162 mg bilberry (25% anthocyanins, 0.081 mmol) and 18 mg (0.149 mmol) L-cysteine](hereinafter “Bil/Cys”) at 37° C. for 30, 60 and 120 minutes. At each time point, 3 wells were processed for analysis by collection of the incubation medium and extraction of lutein absorbed into the cells.

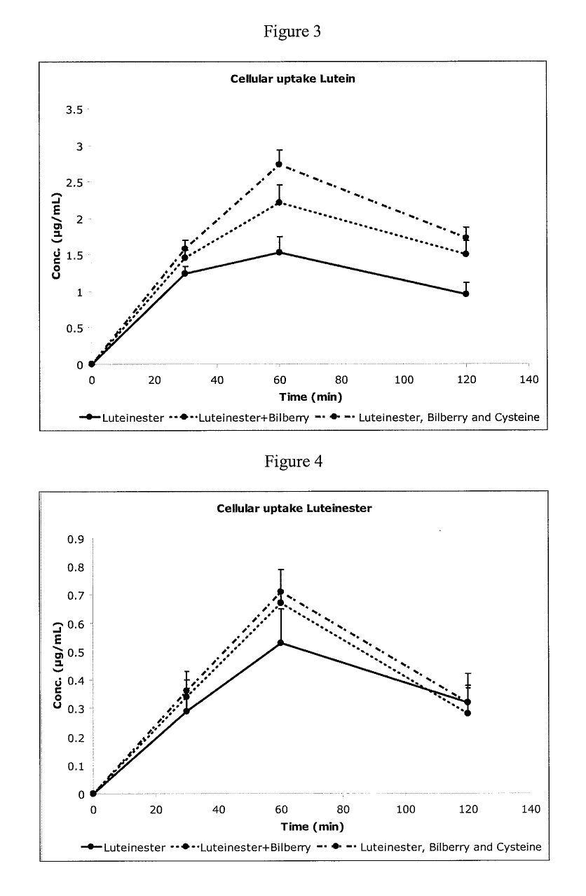

FIG. 3 provides the results for the cellular uptake of lutein from various preparations. Table 4 provides pharmacokinetic results.

TABLE 4
Pharmacokinetic evaluation of cellular uptake of Lutein

| | | Lutein + | Lutein + |
|---|---|---|---|
| | Lutein | Bilberry | Bil/Cys |
| Cₘₐₓ (μg/mL) | 1.53 | 2.22 | 2.74 |
| Total uptake (μg · min/mL) | 135 | 189 | 222 |

3.2 Cellular Uptake of Lutein (Esterified Form) in CaCo-2 Cells

CaCo-2 cells were incubated with 20 mg Luteindipalmitinic-ester/100 mL medium, 20 mg (0.019 mmol) Luteindipalmitinic-ester plus 160 mg bilberry extract (25% anthocyanins, 0.08 mmol)/100 mL medium or 20 mg Luteindipalmitinic-ester plus 180 mg Bil/Cys [equivalent to 162 mg bilberry (25% anthocyanins, 0.081 mmol) and 18 mg (0.149 mmol) L-cysteine] at 37° C. for 30, 60 and 120 minutes. At each time point, 3 wells were processed for analysis by collection of the incubation medium and extraction of Luteindipalmitinic-ester absorbed into the cells. After hydrolysis of the luteinester, lutein was quantified in the cells.

FIG. 4 provides the results for the cellular uptake of Luteindipalmitinic-ester from various preparations. Table 5 provides the corresponding pharmacokinetic results.

TABLE 5
Pharmacokinetic evaluation of cellular uptake of Luteinester

| | | Luteinester + | Luteinester + |
|---|---|---|---|
| | Luteinester | Bilberry | Bil/Cys |
| Cₘₐₓ (μg/mL) | 0.530 | 0.670 | 0.710 |
| Total uptake (μg · min/mL) | 42.2 | 48.8 | 52.4 |

### 4. CONCLUSIONS

4.1 When lutein and anthocynins are combined, the bioavailability of lutein was increased.

4.2 L-Cysteine enhances the effect of anthocyanins. When lutein is mixed with Bil/Cys (anthocyanins & L-Cysteine), the bioavailability of lutein was further increased.

4.3 The in vitro stability experiments supported above conclusions. It was also noted that L-Cysteine alone could also protect lutein.

4.4 The protective effect increased with the dosage of L-Cysteine or Anthocyanins. When the concentration of L-Cysteine reached about 4.1 μmol/ml, an enhanced protective effect was noted. When the concentration of Bilberry extract reached about 0.6 μmol/ml, an enhanced protective effect was noted.

### Cellular Uptake of Lutein and Zeaxanthine in CaCo-2 Cells

### Experimental Methods

### Culturing of CaCo-2 Cells

CaCo-2 cells were cultured in Dulbeccos's Modified Eagle Medium (DMEM) containing 20% fetal bovine serum, 1.2% nonessential amino acids, 0.83 mM L-glutamine, 1.2% penicillin-streptomycin and 0.1% mercaptoethanole in an atmosphere of 5% CO₂ and 95% air at 37° C.

Cells were grown in 75 cm² culture-flasks (T75) and sub-cultured after one week (every other day washed with PBS buffer, removed with trypsin and transferred to a new culture flask).

### CaCo-2 Test

For experiments, cells were seeded in 6 well plates at a density of 3×10⁵ cells per well and grown in an atmosphere of 5% CO₂ and 95% air at 37° C., 7 to 8 days until confluence was reached. The cells were washed with PBS buffer, incubated with 4 ml medium containing the suspended samples for 30, 60 or 120 minutes.

After the corresponding incubation time, the cells were washed with PBS buffer and removed using 1 ml of TBME. Cells were sonicated 3 times for 30 seconds, centrifuged for 10 min and the pellets were discarded. The supernatant was used as sample for HPLC (lutein).

### Pharmacokinetics

Cmax was taken directly from the concentration/time data obtained from the analysis.

The concentration/time data obtained from the analysis were further submitted to non-compartmental calculation of the AUC₀₋₁₂₀ₘᵢₙ using the trapezoidal rule.

### Analysis of Lutein

| | |
|---|---|
| Pump: | Merck Quaternary Gradient pump 6200, Merck |
| | AS 2000, HP-MVD 1050, column oven Tech Lab |
| Column: | YMC C30, 300 × 4.6 mm |
| Mobile phase: | TBME/Methanol = 25/75 (v/v) |
| Flow Rate: | 1 mL/min |
| Temperature: | Ambient |
| Injection volume: | 20 μl |
| Detection: | 450 nm |

### Standard Preparation (Lutein)

An appropriate amount of standard was transferred into a 10 mL flask and dissolved in 1 mL chloroform. The sample was then diluted to the mark with mobile phase. Dilutions were prepared in mobile phase.

### Data Evaluation

Quantification was performed by external standardization after linear regression analysis.

### Experimental Content:

CaCo-2 cells were incubated with (9 mg Lutein+1 mg Zeaxanthine, see Table 6)/100 mL with or without 160 mg black currant extract/100 mL medium. Incubations were performed at 37° C. for 30, 60 and 120 minutes. At each time point, 3 wells were processed for analysis by collection of the incubation medium and extraction of Lutein/Zeaxanthine absorbed into the cells.

In a control experiment, the stability of the analytes under the experimental conditions was investigated.

The content of lutein and zeaxanthine in the sample used for incubation testing was determined.

TABLE 6
Content of Lutein and Zeaxanthine in the test sample submitted

| | Lutein | Zeaxanthine |
|---|---|---|
| Content (g/100 g sample) | 87.0 | 8.9 |
| Content (relative %) | 90.7 | 9.3 |

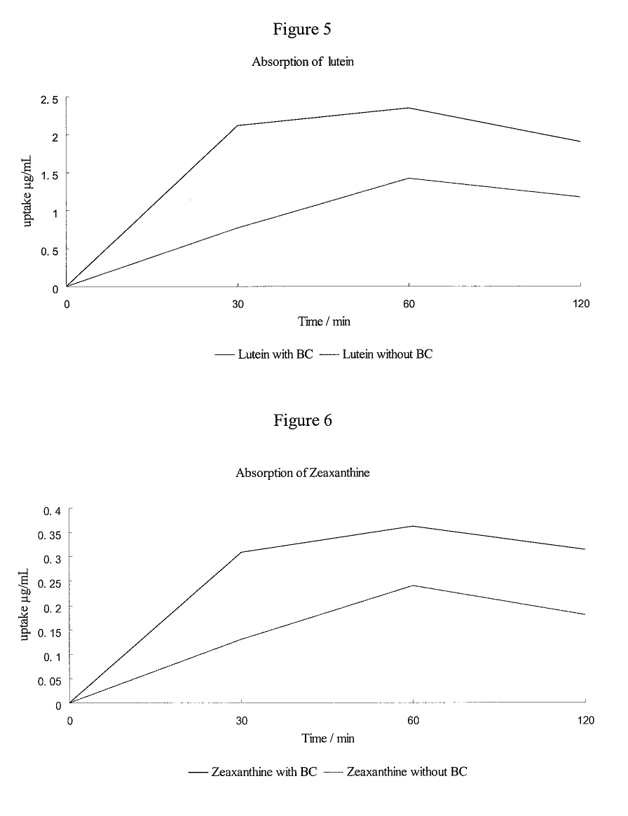

FIGS. 5 and 6 and Table 7 demonstrate results for the cellular uptake of Lutein and Zeaxanthine with or without Black Currant extract. Table 6 provides the corresponding pharmacokinetic results. As seen, the absorption of both, Lutein and Zeaxanthine is increased by roughly a factor of 2 in presence of Black Currant extract.

TABLE 7
Tabulated Plasma levels observed

| | Lutein (μg/mL) | | Zeaxanthine (μg/mL) | |
|---|---|---|---|---|
| Time (min) | with BC | without BC | with BC | without BC |
| 0 | 0 | 0 | 0 | 0 |
| 30 | 2.120 | 0.776 | 0.309 | 0.131 |
| 60 | 2.346 | 1.420 | 0.363 | 0.240 |
| 120 | 1.897 | 1.170 | 0.314 | 0.181 |

When comparing the relative ratio of Lutein and zeaxanthine absorbed the following ratios were obtained (see Table 8). The relative amount of zeaxanthine absorbed indicated a higher absorption of zeaxanthine. While the test sample contained 9.3% zeaxanthine, the level in cells was found to be between 12.7 and 14.5%.

TABLE 8
Comparative, relative cellular uptake of Lutein and Zeaxanthine*

| | Lutein (%) | | | Zeaxanthine (%) | | |
|---|---|---|---|---|---|---|
| | 30 min | 60 min | 120 min | 30 min | 60 min | 120 min |
| With Black | 87.3 | 85.8 | 86.6 | 12.7 | 14.2 | 13.4 |
| Currant | | | | | | |
| Without Black | 85.6 | 85.5 | 87.3 | 14.4 | 14.5 | 12.7 |
| Currant | | | | | | |

*The sum of lutein and Zeaxanthine is set to 100%

The pharmacokinetic parameters calculated are presented in Table 9.

TABLE 9
Pharmacokinetic evaluation of cellular
uptake of Lutein and Zeaxanthine

| | Lutein | | Zeaxanthine | |
|---|---|---|---|---|
| | with BC | without BC | with BC | without BC |
| Cₘₐₓ (μg/mL) | 2.346 | 1.420 | 0.363 | 0.240 |
| AUC₀₋₁₂₀ (μg × min/mL) | 226.1 | 122.3 | 35.01 | 20.17 |

Table 10 provides results for the stability of lutein and zeaxanthine in the incubation medium. As seen, no breakdown of compounds was detected under test assay conditions. The slight increase is most likely caused by evaporation of the medium during 120 minutes at 37° C.

TABLE 10
Stability of Lutein and Zeaxanthine in incubation medium

| Time (min) | Lutein (%) | Zeaxanthine (%) |
|---|---|---|
| 0 | 100 | 100 |
| 120 | 108 | 106 |

In sum, lutein and zeaxanthine showed better absorption in presence of black currant extract. Moreover, the results indicate that zeaxanthine yields a better absorption as compared to lutein.

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A composition comprising a carotenoid and a compound with at least one —SH group.

2. The composition according to claim 1, wherein the composition further comprises an anthocyanin.

3. The composition according to claim 1, wherein the carotenoid is of free form.

4. The composition according to claim 1, wherein the compound with —SH group is L-cysteine or glutathione.

5. The composition according to claim 1, wherein the carotenoid is a xanthophyll or a derivative.

6. The composition according to claim 5, wherein the xanthophyll is lutein, zeaxanthin, capsorubin, capsanthin, astaxanthin, canthaxanthin or mixtures thereof.

7. A method to increase the bioavailability of a carotenoid, comprising the step of combining a compound having at least one —SH group and a carotenoid, such that the bioavailability of the carotenoid is increased relative to a composition without the compound having at least one —SH group.

8. The method of claim 7, wherein the bioavailability of the carotenoid is increased at least about fifteen percent of the amount of bioavailable carotenoid without the compound having at least one —SH group.

9. The method of claim 7, wherein the bioavailability of the carotenoid is increased at least about fifteen percent to about 4 times of the amount of bioavailable carotenoid without the compound having at least one —SH group.

10. A method to increase the bioavailability of a carotenoid, comprising the step of combining an anthocyanin and a carotenoid, such that the bioavailability of the carotenoid is increased relative to a composition without the anthocyanin.

11. The method of claim 10, wherein the bioavailability of the carotenoid is increased at least about fifteen percent of the amount of bioavailable carotenoid without the anthocyanin.

12. The method of claim 10, wherein the bioavailability of the carotenoid is increased at least about fifteen percent to about 4 times of the amount of bioavailable carotenoid without the anthocyanin.

13. A method to increase the bioavailability of a carotenoid, comprising the step of combining an anthocyanin, a compound having at least one —SH group and a carotenoid, such that the bioavailability of the carotenoid is increased relative to a composition without the anthocyanin and compound having at least one —SH group.

14. The method of claim 13, wherein the bioavailability of the carotenoid is increased at least about fifteen percent of the amount of bioavailable carotenoid without the anthocyanin and compound having at least one —SH group.

15. The method of claim 13, wherein the bioavailability of the carotenoid is increased at least about fifteen percent to about 4 times of the amount of bioavailable carotenoid without the anthocyanin and compound having at least one —SH group.

16. The method according to claim 7, wherein the carotenoid is of free form.

17. The method according to claim 10, wherein the carotenoid is of free form.

18. The method according to claim 13, wherein the carotenoid is of free form.

19. The method according to claim 7, wherein the carotenoid is lutein, zeaxanthin, capsorubin, capsanthin, astaxanthin, canthaxanthin or mixtures thereof.

20. The method according to claim 10, wherein the carotenoid is lutein, zeaxanthin, capsorubin, capsanthin, astaxanthin, canthaxanthin or mixtures thereof.

21. The method according to claim 13, wherein the carotenoid is lutein, zeaxanthin, capsorubin, capsanthin, astaxanthin, canthaxanthin or mixtures thereof.

22. The composition of claim 2, wherein the anthocyanin is a mixture of anthocyanins present in bilberry extract.

23. The method of claim 10, wherein the anthocyanin is a mixture of anthocyanins present in bilberry extract.

24. A composition comprising a carotenoid and an anthocyanin, wherein the anthocyanin is present in an amount sufficient to increase the bioavailability of the carotenoid at least about fifteen percent of the amount of bioavailable carotenoid without the anthocyanin.

25. The composition according to claim 24, wherein the carotenoid is of free form.

26. The composition according to claim 25, wherein the carotenoid is a xanthophyll or a derivative.

27. The composition according to claim 26, wherein the xanthophyll is lutein, zeaxanthin, capsorubin, capsanthin, astaxanthin, canthaxanthin or mixtures thereof.

28. The composition of claim 24, wherein the anthocyanin is a bilberry extract or a black currant extract.
